(19) United States
(12) Patent Application Publication
(10) Pub. No.: US 2025/0229249 A1
(43) Pub. Date: Jul. 17, 2025

(54) NEW PHOTOCHEMICAL REACTOR DESIGN AND PROCESS FOR PERFORMING EXOTHERMIC PHOTOCHEMICAL REACTIONS

(71) Applicant: FLUORINNOVATION L.L.C-FZ, Nad Al Sheba Dubai (AE)

(72) Inventor: Max Josef Braun, Wedemark (DE)

(73) Assignee: FLUORINNOVATION L.L.C-FZ, Nad Al Sheba Dubai (AE)

(21) Appl. No.: 19/170,165

(22) Filed: Apr. 4, 2025

Related U.S. Application Data
(63) Continuation of application No. PCT/IB2023/059917, filed on Oct. 3, 2023.

(30) Foreign Application Priority Data
Oct. 5, 2022 (DE) 10 2022 125 708.8

(51) Int. Cl. B01J 19/12; B01J 19/24; C07C 51/21

(52) U.S. Cl. CPC B01J 19/123; B01J 19/2415; C07C 51/21; B01J 2219/0254; B01J 2219/1203

(57) ABSTRACT

A photochemical reactor and a photo-oxidation process for manufacturing organic compounds are disclosed. The photo-oxidation generates polyfluorochloro- and/or perfluorocarboxylic acid chloride compounds, in particular halogenated acetyl chlorides, such as polyfluorochloro- and/or perfluoro acetyl chloride. The photo-oxidation process is exothermic. The reactor has a channel or a channels system, including tubes, pipes, or coils, such as coiled tubes or pipes, and, optionally, gasket installations in combination with tubes/pipes, and interior installations, such as microtubes or micropipes. The interior installations avoid formation of hot spots and provide for very good mixing of the reaction medium, such that the photochemical reactions do not only take place in part of reaction medium within a few mm around the UV lighting source. Formation of oxidizing by-products is eliminated, which may otherwise be formed during the photochemical oxidation and possibly accumulate in the photochemical reactor and/or process equipment, such as cooling traps and piping systems.

### CROSS REFERENCE TO RELATED APPLICATIONS

This application is a continuation application of international patent application PCT/IB2023/059917, filed on Oct. 3, 2023 and designating the U.S., which claims priority to German patent application DE 10 2022 125 708.8, filed on Oct. 5, 2022, both of which are hereby incorporated by reference in their entireties.

### TECHNICAL FIELD

The disclosure pertains to a novel photochemical reactor design, to a novel process for the manufacture of organic compounds by photo-oxidation using said novel photochemical reactor design, and in a further aspect to a related novel process for the manufacture of organic compounds by photo-oxidation using a photochemical microreactor. In another aspect, the disclosure pertains to a process for the manufacture of polyfluorochloro- and/or perfluorocarboxylic acid chloride compounds by photo-oxidation, and in particular of halogenated acetyl chlorides, e.g., such as polyfluorochloro- and/or perfluoro acetyl chloride. In particular, the photo-oxidation process of the disclosure for the manufacture of said organic compounds is exothermic. In a further aspect the disclosure also pertains to a novel process for the elimination of oxidizing by-products which possibly may be formed during a photochemical oxidation and possibly accumulate at certain parts and/or certain positions of a photochemical reactor system and/or process equipment used in context of a photochemical oxidation, for example, such equipment as cooling traps and piping systems.

### BACKGROUND

Halogenated acetyl chlorides are important starting materials for many active ingredients for pharma- as well as agro applications.

Among those active ingredients for pharma- as well as agro applications, there are compounds with a CF₃-group, e.g., the insecticide like the already generic Flonicamid, the fungizide Penthiopyrad, the herbicide Bicyclopyrone, and the diabetes drug Sitagliptin. Other active ingredients for pharma- as well as agro applications have a CF₂-group, e.g., there are quite new representatives within a new fungizide family like the Bixafen, Fluxapyroxad Benzovindiflupyr and Sedaxane (mixture of four isomers, only the cis-compound is shown). Some other active ingredients for pharma- as well as agro applications having a CCl₂-group or a CCl₃-group, respectively, are also on the market.

In general, compounds having a CCl₃-group can serve as starting materials for more important CF₃-compounds, i.e., active ingredients for pharma- as well as agro applications having a CF₃-group.

Representatives of such active ingredients for pharma- as well as agro applications are exemplified in the herein after by their chemical formulae:

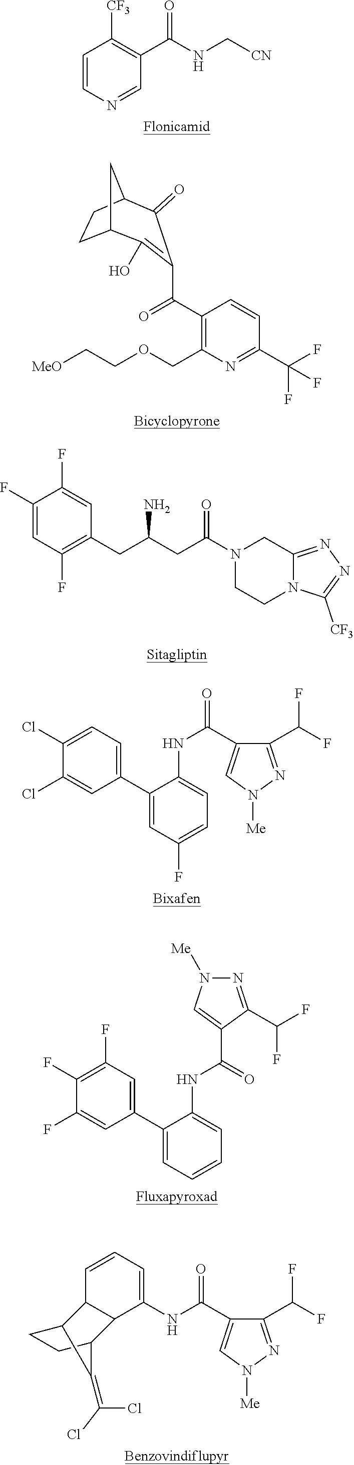 [SMILES: N#CCNC(=O)c1cnccc1C(F)(F)F ; COCCOCc1nc(C(F)(F)F)ccc1C(=O)C1=C(O)C2CCC(C2)C1=O ; N[C@@H](CC(=O)N1CCn2c(nnc2C(F)(F)F)C1)Cc1cc(F)c(F)cc1F ; Cn1cc(C(=O)Nc2ccc(F)cc2-c2ccc(Cl)c(Cl)c2)c(C(F)F)n1 ; Cn1cc(C(=O)Nc2ccccc2-c2cc(F)c(F)c(F)c2)c(C(F)F)n1 ; Cn1cc(C(=O)NC2=CC=CC3C4CCC(C4=C(Cl)Cl)C23)c(C(F)F)n1]

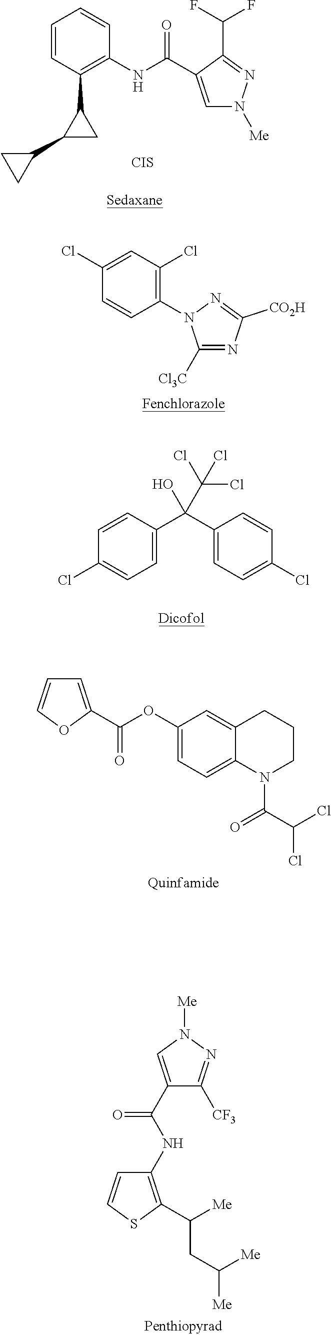

The newest launched active ingredient containing a CF₃-group is Pfizer's SARS-CoV-2 drug PF-07321332 launched under the trade name Paxlovid, and with chemical structure given hereunder:

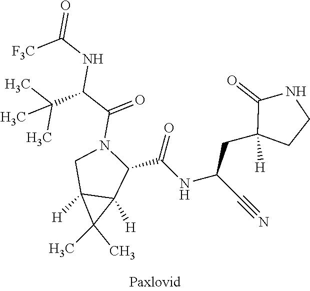 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C]

There is still a further new very successful active ingredient (fungicides from Syngenta), namely Pydiflumetofen. The synthesis of Pydiflumetofen uses chlorodifluoroacetylchloride (CDFAC) as starting material.

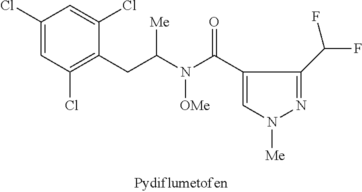 [SMILES: CON(C(=O)c1cn(C)nc1C(F)F)C(C)Cc1c(Cl)cc(Cl)cc1Cl]

For all such active ingredients (just a few examples are given above), e.g., having a CF₃-group, CF₂-group, CCl₂-group or CCl₃-group, respectively, a late stage fluorination, which means a direct fluorination, e.g., with HF, F₂ or another fluorinating agent, respectively, is not possible at all due to the presence of functional groups or other reactive groups, or such late stage fluorination either is possible only with quite low yields or is possible only with fluorinating agents suitable for small-scale investigations (examples of such so-called small-scale fluorinating agents are compounds like Deoxofluor, Ruppert's reagent, Selectfluor, XtalFluor), but which are expensive, cause separation problems and last but not least in large industrial-scale environmental problems because only a small ratio of the molecular weight is used as fluorinating “F” and because the F-carrier residue needs to be incinerated.

Therefore, the chemically most comfortable and environmentally friendly synthesis pathway for manufacturing said active ingredients having a CF₃-group, CF₂-group, CCl₂-group or CCl₃-group, respectively, is the so-called building block approach wherein C—F-bond(s) and/or C—Cl-bond(s) is (are) already present in a building block used as starting material to synthesize the before said active ingredients.

Most convenient starting materials building block(s), respectively, for direct usage or usage as a derivative thereof are above said halogenated carboxylic acid chlorides, i.e., the polyfluorochloro- and/or perfluorocarboxylic acid chloride compounds.

Particular examples of such halogenated carboxylic acid chlorides, i.e., the polyfluorochloro- and/or perfluorocarboxylic acid chloride compounds, are trifluoroacetyl chloride (TFAC), chlorodifluoroacetylchloride (CDFAC), trichloroacetylchloride (TCAC) and dichloroacetylchloride (DCAC). However the synthesis of these exemplified halogenated carboxylic acid chlorides in the current related art still has disadvantages.

For example, as disclosed in CN 103524325, at present trifluoroacetyl fluoride (TFAF) is prepared in a thermal fluorination process out of expensive 1,1-difluorochloroethane by fluorination followed by oxidation with SO₃; subsequent hydrolysis of obtained TFAF to trifluoroacetic acid (TFA) is also disclosed in CN 103524325. Older processes for preparing trifluoroacetyl fluoride (TFAF) are described by Hoechst in EP0691322 using a chromium/magnesium based fluorination catalyst in a gas phase for conversion of trichloroacetyl chloride (TCAC) with HF to TFAF. In Rhodia's patent application US2012/0190892, TCAC is produced by “short” wavelength photo-oxidation of perchloro ethylene using an Hg-lamp as lightening source followed by fluorination with HF in liquid phase to trichloroacetyl fluoride, and final fluorination to TFAF in gas phase with a Cr-based catalyst like already disclosed by Hoechst (see EP0691322) and also by SRF India in IN2008DE01665.

The most clean and environmentally suitable technology in regard to preparing carboxylic acid chlorides is the preparation of trifluoroacetyl chloride, chloridifluoroacetyl chloride, trichloroacetyl chloride and dichloroacetyl chloride by so-called light induced photo-oxidation.

In respect of so-called light induced photo-oxidation there are two mechanistic types of photo-oxidation:
a) Direct gas phase photo-oxidation with “short” wavelength which is, for example, (mainly) the 254 nm line of an Hg-lamp or other available UV lighting source, equipment made out of quartz glass needs to be used to allow transmission of “short” wavelength to the reaction zone (starting material is activated directly), i.e., where the photo-oxidation takes place. There is a very high demand of photons.
b) A sensitized gas phase photo-oxidation using, e.g., elemental chlorine as light absorber (needed wavelength >290 nm); herein the generated chlorine radicals induce a radical chain photo-oxidation reaction which results in an energy saving as compared to before said “type a” direct gas phase photo-oxidation, and in most cases this “type b” sensitized gas phase photo-oxidation allows for higher yields and/or selectivity. This “type b” sensitized gas phase photo-oxidation results in a lower demand of photons (more energy saving).

Said “type a” direct gas phase photo-oxidation and said “type b” sensitized gas phase photo-oxidation are already known in the related art.

“Type a” photo-oxidation was already mentioned by Hazeldine et al. in the 50ies, and it was already used and improved by Halocarbon in USA as described in U.S. Pat. No. 3,883,407 (year 1976).

Regarding “type b” photo-oxidation, for the first time Solvay Fluor has disclosed in EP659729 a chlorine gas sensitized photo-oxidation of HCFC-123 (2,2-dichloro-1,1,1-trifluoroethane) in gas phase using wavelength >290 nm. In a newer Chinese patent application CN101735034 (Fujian Shunyue Science and Technology Co., Ltd.) also only a procedure “type b” is disclosed which is only suitable for synthesis of compounds in laboratory quantities, and which is a modification of Solvay Fluor's process disclosed in EP659729.

The related art processes of photo-oxidation in general, for example, have the following disadvantages: UV Hg-lamp reactor with immersion lamp (UV lighting source) of related art has insufficient mixing, the reaction zone is only very nearby the lamp (UV lighting source) which can lead to over-oxidized products (phosgenes, CO₂) and low/no reaction farer away from the lamp (UV lighting source); tube reactor/UV Hg-lamp reactor with immersion lamp (UV lighting source) of related art has insufficient heat control which results in hot spots in regard to very exothermic photo-oxidation reaction; hot spots in the reactor, if not avoided, decrease selectivity; insufficient mixing capability of related art reactors decrease conversion (productivity) and results in higher energy/electricity consumption.

Furthermore, although photo-oxidation reactions are very clean-type light induced reactions with high yields and high selectivity, agent molecular oxygen may cause some disadvantages in photochemical oxidations especially such ones performed at lower temperatures, because of potential formation of undesired “oxidizing material” or “oxidizing by-products,” respectively. For example, such undesired “oxidizing material” or “oxidizing by-products” can be found in a photochemical reactor system, e.g., as higher boiling material (regarding term “higher boiling” see explanation and definition, respectively, herein after) and which possibly accumulate at certain positions of photochemical reactor system and/or process equipment used in context of a photochemical oxidation, especially at positions where some potential condensation can occur. For example, such positions at which such condensation can occur can be found in cooling traps or in a pilot plant or industrial unit at positions of the piping system. Especially, for the reason that such potential condensation is depending on dew point of the entire photochemical reaction mixture. Said oxidizing material or oxidizing by-products, respectively, is found to be very corrosive, and thus there is a high need to eliminate such undesired “oxidizing material” or “oxidizing by-products” from a photochemical reactor system and/or other parts and/or other positions of a photochemical reactor system and/or process equipment used in context of a photochemical oxidation where it has formed and/or accumulated.

Regarding the undesired “oxidizing material” or “oxidizing by-products” that can be found in a photochemical reactor system, e.g., as higher boiling material, the term “higher boiling” in the given context means an undesired by-product having a boiling point higher than the targeted product of the photochemical oxidation reaction; for example, the “higher boiling” point is at least 1 to 5 C° higher than the boiling point of the targeted product, typically at least 1 to 5 C.° higher. Without being wished to be bound by theory, here it is assumed that short-chain intermediates (peroxides) normally decompose by themselves because they are only intermediates that react to form the more stable products. In case semi-stable peroxides, e.g., as an intermediate stage, are formed under the reaction conditions, as assumed here, by dimerization of intermediate stages, problems exist such as corrosion and safety problems in the event of uncontrolled decomposition with the release of heat.

Regarding equipment, especially regarding the design of photo-oxidation reactors, the apparatus or reactors used in the related art processes of photo-oxidation, for example, have the following disadvantages:

Especially, when performing exothermic photochemical reactions, there is a high need to overcome the following disadvantages, for example: if hot spots are not avoided, over-oxidation and high energy consumption are the consequence; insufficient mixing capability of a photo reactor leads to low conversion and high energy consumption.

Hence, there is still a demand to improve the photo-oxidation processes and/or the photo-oxidation apparatus or photo-oxidation apparatus, respectively, in general, and in particular regarding performing exothermic photochemical reactions of organic compounds. Also, there is still a high demand to provide such photo-oxidation processes and/or the photo-oxidation apparatus or photo-oxidation apparatus, respectively, in general, and in particular regarding performing exothermic photochemical reactions, which are suitable for industrial scale photo-oxidation of organic compounds.

### SUMMARY

It is an object of the present disclosure to overcome the disadvantages of the related art photo-oxidation processes and/or the photo-oxidation apparatus or photo-oxidation apparatus, respectively, in particular to provide more efficient and energy saving photo-oxidation processes, also a more environmentally friendly photo-oxidation process, for the manufacture of organic compounds by photo-oxidation and/or by industrial scale photo-oxidation, in particular by an exothermic photochemical reaction and/or industrial scale exothermic photo-oxidation.

It is also an object of the present disclosure to provide a novel photochemical reactor (apparatus) design for performing exothermic photochemical reactions, and in particular for performing exothermic photochemical reactions, in the manufacture of organic compounds. A further object is that the novel photochemical reactor (apparatus) design for performing exothermic photochemical reactions allows for a more environmentally friendly photo-oxidation process, for the manufacture of organic compounds by photo-oxidation and/or by industrial scale photo-oxidation, in particular for performing an exothermic photochemical reaction and/or industrial scale exothermic photo-oxidation.

In still another object of the present disclosure it is desired to provide a photo-oxidation processes and/or the photo-oxidation apparatus or photo-oxidation apparatus, respectively, which enable(s) for a chemically more comfortable and more environmentally friendly synthesis pathway for manufacturing active ingredients for pharma- and/or agro applications, for example, such active ingredients as referenced above, having a CF₃-group, CF₂-group, CCl₂-group or CCl₃-group, respectively, and to provide a building block required for the for manufacturing said active ingredients for pharma- and/or agro applications, for example, such active ingredients as referenced above, wherein in the building block C—F-bond(s) and/or C—Cl-bond(s) is (are) already present in the building block required as starting material to synthesize a before said active ingredient.

Accordingly, in one aspect it is also an object of the present disclosure to provide convenient starting materials building block(s), respectively, for direct usage or usage as a derivative thereof, for manufacturing active ingredients for pharma- and/or agro applications, for example, such active ingredients as referenced above, having a CF₃-group, CF₂-group, CCl₂-group or CCl₃-group, respectively, which are above said halogenated carboxylic acid chlorides, i.e., the polyfluorochloro- and/or perfluorocarboxylic acid chloride compounds.

Accordingly, in a further aspect it is also an object of the present disclosure to provide convenient starting materials building block(s), respectively, for direct usage or usage as a derivative thereof, for manufacturing active ingredients for pharma- and/or agro applications, for example, such active ingredients as referenced above, having a CF₃-group, CF₂-group, CCl₂-group or CCl₃-group, respectively, which are halogenated carboxylic acid chlorides, i.e., the polyfluorochloro- and/or perfluorocarboxylic acid chloride compounds, are trifluoroacetyl chloride (TFAC), chlorodifluoroacetylchloride (CDFAC), trichloroacetylchloride (TCAC) and dichloroacetylchloride (DCAC). However the synthesis of said exemplified halogenated carboxylic acid chlorides in the current related art still has disadvantages.

In still a further aspect it is an object of the present disclosure to provide convenient process for eliminating any undesired, in particular corrosive, oxidizing material or oxidizing by-products, such as addressed further above, from a photochemical reactor system and/or other parts and/or other positions of a photochemical reactor system and/or process equipment used in context of a photochemical oxidation where it has formed and/or accumulated.

### BRIEF DESCRIPTION OF DRAWINGS

FIG. 1 schematically shows a first photochemical reactor;

FIG. 2 schematically shows an alternative photochemical reactor;

FIG. 3 schematically shows another alternative photochemical reactor;

FIG. 4 schematically shows yet another alternative photochemical reactor;

FIGS. 5A and 5B show reactor designs and photochemical reactions performed therein;

FIG. 6 shows a representative scheme of a photochemical microreactor;

FIG. 7 shows a representative scheme of an alternative photochemical microreactor;

FIG. 8 shows microreactor designs and photochemical reactions performed therein; and

FIGS. 9A and 9B show photochemical reactions with a continuous decomposition procedure.

### DESCRIPTION OF EXEMPLARY EMBODIMENTS

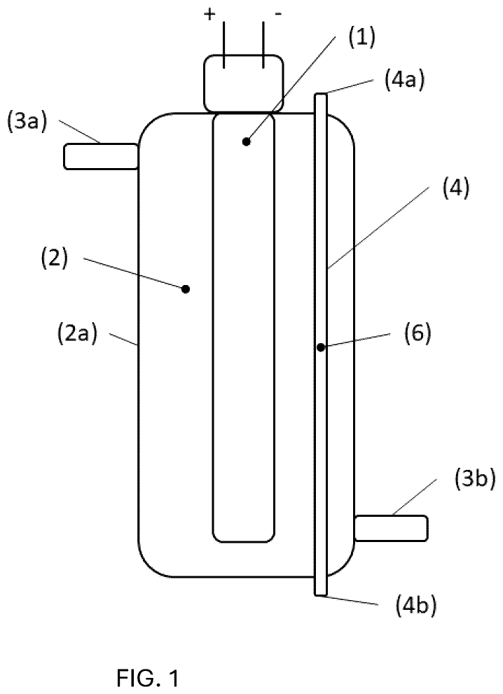

FIG. 1 shows a representative scheme of a photochemical reactor with interior installations, comprising at least one UV lighting source (1) providing UV lighting emission in the range of short-wave UV to long-wave UV (details of lamp and lamp tube not drawn); a reactor reservoir (2), surrounded by reactor wall (2a); one of one or more inlet(s)/outlet(s) installation(s) (3a) and (3b); and one of one or more interior installations (4) each forming a channel (6) inside the reactor reservoir (2), e.g., in vertical arrangement or in parallel arrangement, respectively, to the at least one UV lighting source (1); and wherein the one or more interior installations (4) are in tube/pipe form (e.g., forming inside a channel (6)) and consist out of borosilicate glass, and wherein each of the interior installations (4) at both tube/pipe ends have an inlet/outlet installation (4a) and (4b) extending to the exterior of the reactor reservoir (2).

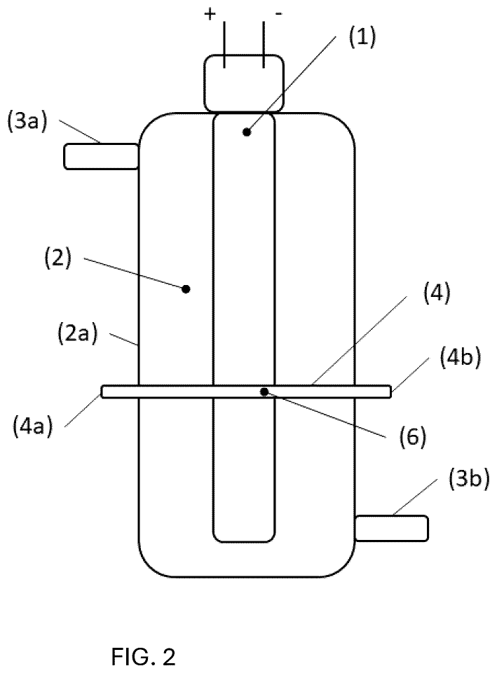

FIG. 2 shows another representative scheme, alternative to that shown in FIG. 1, of a photochemical reactor with interior installations, comprising at least one UV lighting source (1) providing UV lighting emission in the range of short-wave UV to long-wave UV (details of lamp and lamp tube not drawn); a reactor reservoir (2), surrounded by reactor wall (2a); one of one or more inlet(s)/outlet(s) installation(s) (3a) and (3b); and one of one or more interior installations (4) each forming a channel (6) inside the reactor reservoir (2), e.g., in horizontal arrangement, or in right angle arrangement to the at least one UV lighting source (1), respectively; and wherein the one or more interior installations (4) are in tube/pipe form (e.g., forming inside a channel (6)) and consist out of borosilicate glass, and wherein each of the interior installations (4) at both tube/pipe ends have an inlet/outlet installation (4a) and (4b) extending to the exterior of the reactor reservoir (2).

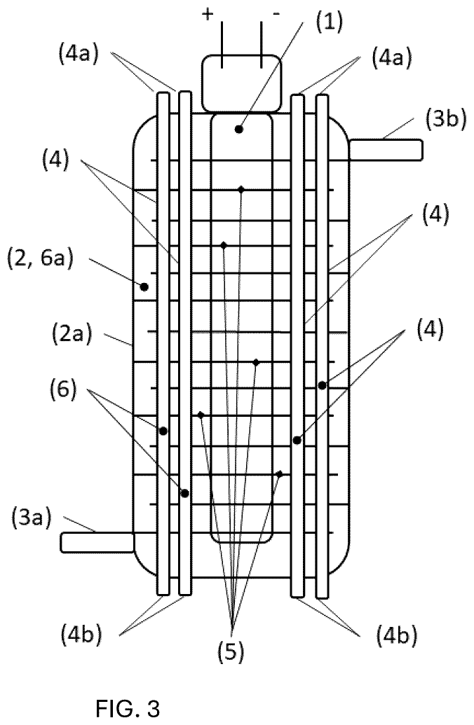

FIG. 3 shows a representative scheme of a photochemical reactor with interior installations, comprising at least one UV lighting source (1) providing UV lighting emission in the range of short-wave UV to long-wave UV (details of lamp and lamp tube not drawn); a reactor reservoir (2), surrounded by reactor wall (2a); one of one or more inlet(s)/outlet(s) installation(s) (3a) and (3b); and one of one or more interior installations (4) each forming a channel (6) inside the reactor reservoir (2), e.g., in vertical arrangement, or in parallel arrangement to the at least one UV lighting source (1), respectively (e.g., like shown in FIG. 1, too); wherein the one or more interior installations (4) are in tube/pipe form (e.g., forming inside a channel (6)) and consist out of borosilicate glass, and wherein each of the interior installations (4) at both tube/pipe ends have an inlet/outlet installation (4a) and (4b) extending to the exterior of the reactor reservoir (2); and further comprising at least two or more interior installations (5) inside the reactor reservoir (2), wherein the two or more interior installations (5) divide the reactor reservoir (2) into a further channel system (6a), each channel (6a) starting/ending at one of the one or more inlet(s)/outlet(s) installation(s) (3a) and (3b), and wherein each of said channel (6a) formed by said two or more interior installations (5) are in horizontal arrangement, or in right angle arrangement, respectively, to the at least one UV lighting source (1). Thus, said two or more interior installations (5) forming the further channel system (6a) inside the reactor reservoir (2) forces a reaction medium to pass along the outer wall side(s) of said one or more interior installations (4) in tube/pipe form; e.g., which one or more interior installations (4) in tube/pipe form are heat exchanger pipes with inside a cooling liquid flowing through channel (6). Reference is made to Examples 3, 5, 6 and 7 of the disclosure. The number of the interior installations (5) depends on the size, e.g., of the height of the reactor reservoir (2), as further described herein below.

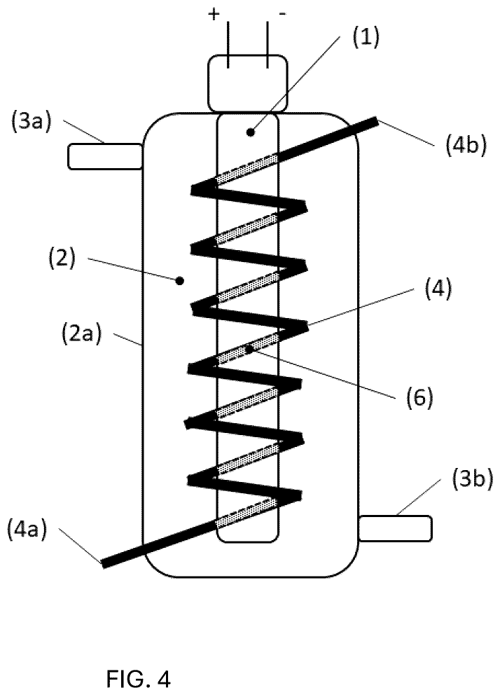

FIG. 4, is showing a further representative scheme, alternative to that shown in FIG. 1, of a photochemical reactor with interior installations, comprising at least one UV lighting source (1) providing UV lighting emission in the range of short-wave UV to long-wave UV (details of lamp and lamp tube not drawn); a reactor reservoir (2), surrounded by reactor wall (2a); one of one or more inlet(s)/outlet(s) installation(s) (3a) and (3b); and one of one or more interior installations (4) each forming a channel (6) inside the reactor reservoir (2), e.g., in vertical arrangement or in parallel arrangement, respectively, to the at least one UV lighting source (1); and wherein the one or more interior installations (4) are in tube/pipe form (e.g., forming inside a channel (6)) are coiled around the at least one UV lighting source (1), e.g., are in a coil reactor form (e.g., forming inside a channel (6)), and consist out of borosilicate glass, and wherein each of the interior installations (4) at both tube/pipe ends or at both coil reactor ends, respectively, have an inlet/outlet installation (4a) and (4b) extending to the exterior of the reactor reservoir (2). Thus, said one or more interior installations (4) in tube/pipe form coiled around the at least one UV lighting source (1), e.g., in coil reactor form, and e.g., forming a channel (6) inside the reactor reservoir (2) (e.g. comprising therein a cooling liquid), forces a reaction medium to pass along said at least one UV lighting source (1). Reference is made to Examples 9 and 10 of the disclosure.

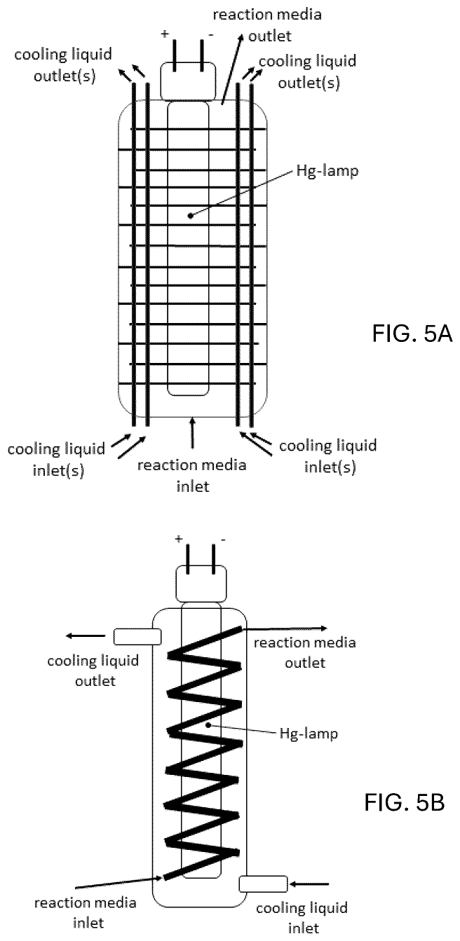

FIGS. 5A and 5B are showing a representative schemes of reactor designs and photochemical reactions performed therein, according to the examples of the disclosure.

Herein, FIG. 5A is showing a first scheme, wherein during the photochemical oxidation the interior installations in the reactor forces reaction medium to pass along heat exchanger pipes (details of lamp and lamp tube not drawn); see also FIG. 3. Reference is made to Examples 3, 5, 6 and 7 of the disclosure.

Herein, FIG. 5B is showing a second scheme, with a simplified coil reactor in cooling liquid around the Hg-lamp (details of lamp and lamp tube not drawn) within the reactor reservoir, wherein during the photochemical oxidation the channel of the interior installations in form of a coil in the reactor (which is coiled around the Hg-lamp) forces reaction medium to pass along the Hg-lamp; see also FIG. 4. Reference is made to Examples 9 and 10 of the disclosure.

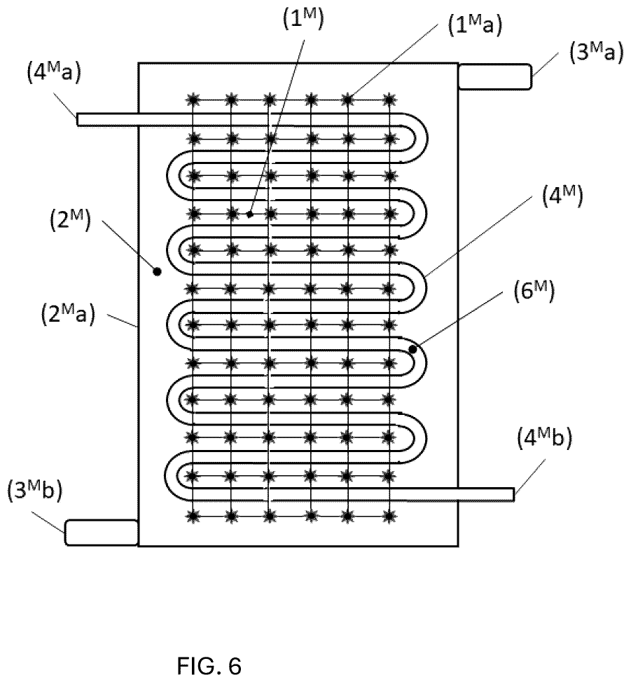

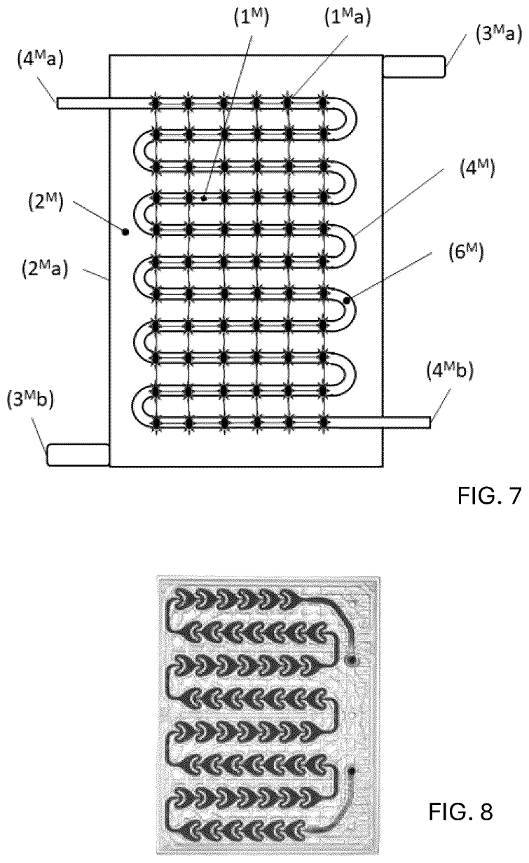

FIG. 6 shows a representative scheme of a photochemical microreactor with interior installations, comprising at least one array (1^{M}) of UV lighting sources (1^{M}a) providing UV lighting emission in the range of short-wave UV to long-wave UV (details of lamp and lamp tube not drawn); a microreactor reservoir (2^{M}), surrounded by microreactor wall (2^{M}a); one of one or more inlet(s)/outlet(s) installation(s) (3^{M}a) and (3^{M}b); and one of one or more interior installations (4^{M}) each forming a microchannel (6^{M}) inside the microreactor reservoir (2^{M}), e.g., in in parallel arrangement to the at least one array (1^{M}) of UV lighting sources (1^{M}a); and wherein the one or more interior installations (4^{M}) are in tube/pipe form (e.g., forming inside a microchannel (6^{M})) and consist out of borosilicate glass, and wherein each of the interior installations (4^{M}) at both tube/pipe ends have an inlet/outlet installation (4^{M}a) and (4^{M}b) extending to the exterior of the reactor reservoir (2^{M}). See also FIG. 7 for alternative arrangement of the array of LEDs.

FIG. 7, is showing another representative scheme, alternative to that shown in FIG. 6, of a photochemical microreactor with interior installations, comprising at least one array (1^{M}) of UV lighting sources (1^{M}a) providing UV lighting emission in the range of short-wave UV to long-wave UV (details of lamp and lamp tube not drawn); a microreactor reservoir (2^{M}), surrounded by microreactor wall (2^{M}a); one of one or more inlet(s)/outlet(s) installation(s) (3^{M}a) and (3^{M}b); and one of one or more interior installations (4^{M}) each forming a microchannel (6^{M}) inside the microreactor reservoir (2^{M}), e.g., in in parallel arrangement to the at least one array (1^{M}) of UV lighting sources (1^{M}a); and wherein the one or more interior installations (4^{M}) are in tube/pipe form (e.g., forming inside a microchannel (6^{M})) and consist out of borosilicate glass, and wherein each of the interior installations (4^{M}) at both tube/pipe ends have an inlet/outlet installation (4^{M}a) and (4^{M}b) extending to the exterior of the reactor reservoir (2^{M}); and wherein the at least one array (1^{M}) of UV lighting sources (1^{M}a) is integrated to the one or more interior installations (4^{M}) in tube/pipe form (e.g., forming inside a microchannel (6^{M})). Reference is made to Reference is made to Examples 11 and 12 of the disclosure.

FIG. 8 is showing a representative scheme of photochemical microreactor designs and photochemical reactions performed therein, according to the examples of the disclosure. Herein, FIG. 8 is showing a scheme of a commercially available photochemical microreactor design with an array of UV lighting sources integrated to the interior installations in tube/pipe form, e.g., forming inside a microchannel, wherein during the photochemical oxidation the interior installations in form of microchannel in the reactor forces reaction medium to pass along the UV lighting sources of the array of UV lighting sources integrated to the interior microchannel installations; see also FIG. 7. This FIG. represents, for example, a Dow Corning photo reactor type G1, e.g., with an internal volume of 9 ml. The construction shows the UV irradiated glass channels (365 nm LEDs), with a LED power, e.g., of 100 mW/cm². Reference is made to Examples 11 and 12 of the disclosure.

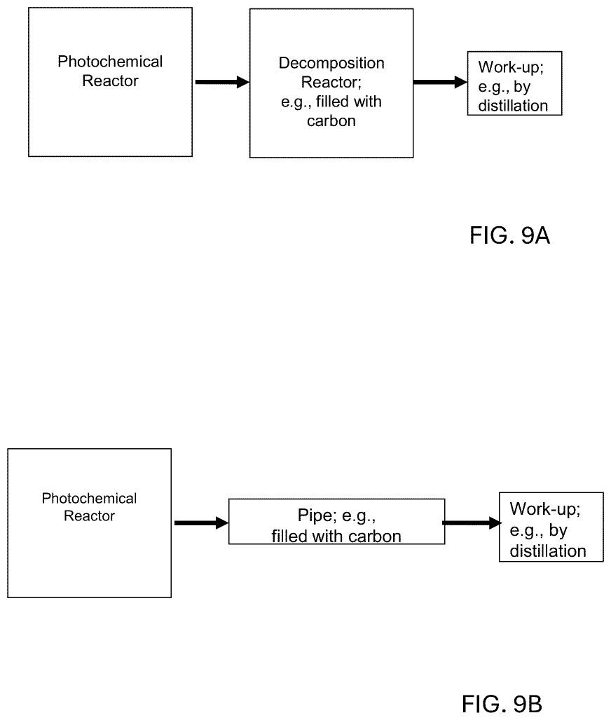

FIGS. 9A and 9B show a representative scheme of a photochemical reaction with a continuous decomposition procedure exemplified for the manufacture of TFAC. FIG. 9A is showing a reactor sequence with a decomposition reactor; e.g., filled with carbon. FIG. 9B is showing a reactor sequence with a pipe as decomposition reactor; e.g., filled with carbon. As a guideline, the size (volume) of the decomposition reactor (for the decomposition of the peroxides) is 1/10 in relation to the reactor volume for the photo-oxidation. Reference is made to Example 21 of the disclosure.

The objects of the disclosure are solved by the photo-oxidation reactor and photo-oxidation processes as described herein.

In a first aspect the present disclosure pertains to a novel photo-oxidation apparatus or photo-oxidation apparatus, respectively, designed for a more efficient and energy saving and/or a more environmentally friendly manufacture of organic compounds by photo-oxidation and/or by industrial scale photo-oxidation, and in particular by an exothermic photochemical reaction and/or industrial scale exothermic photo-oxidation, wherein the novel photo-oxidation apparatus or photo-oxidation apparatus, respectively, has one or more interior installations (4) each forming a channel (6) inside the reactor reservoir (2), wherein the one or more interior installations (4) are in tube/pipe form and consist out of borosilicate glass, and wherein each of the interior installations (4) at both tube/pipe ends have an inlet/outlet installation (4a) and (4b) extending to the exterior of the reactor reservoir (2). Reference is made to the FIGS. 1 to 4, showing representative photo-oxidation apparatus or photo-oxidation apparatus, respectively, of the disclosure which is characterized by the novel concept of one or more interior installations (4) each forming a channel (6) inside the reactor reservoir (2). Reference is also made to FIG. 5B showing a scheme with a simplified coil reactor, e.g., wherein the one or more interior installations (4) each forming a channel (6) are in a coiled form and placed in cooling liquid around the Hg-lamp within the reactor reservoir (2).

Optionally, said novel photo-oxidation apparatus or photo-oxidation apparatus, respectively, of the first aspect the present disclosure has also at least two or more interior installations (5) inside the reactor reservoir (2), wherein the two or more interior installations (5) divide the reactor reservoir (2) into a further channel system (6a), each channel starting/ending at one of the one or more inlet(s)/outlet(s) installation(s) (3a) and (3b). Reference is made to the FIG. 3, showing representative photo-oxidation apparatus or photo-oxidation apparatus, respectively, of the disclosure which is characterized by the novel concept of one or more interior installations (4) each forming a channel (6) inside the reactor reservoir (2), as described here before, and which in addition has an interior installations (5) inside the reactor reservoir (2), wherein the two or more interior installations (5) divide the reactor reservoir (2) into a further channel system (6a). Reference is also made to FIG. 5A showing a scheme of a reactor design wherein during the photochemical oxidation the interior installations in the reactor forces reaction medium to pass along heat exchanger pipes. The number of the interior installations (5) depends on the size, e.g., of the height of the reactor reservoir (2), as further described herein below.

In a second aspect the present disclosure pertains to a novel photo-oxidation processes, in particular a more efficient and energy saving photo-oxidation processes and/or a more environmentally friendly photo-oxidation process, for the manufacture of organic compounds by photo-oxidation and/or by industrial scale photo-oxidation, and in particular by an exothermic photochemical reaction and/or industrial scale exothermic photo-oxidation, and wherein a light induced photochemical oxidation, typically an exothermic light induced photochemical oxidation, is performed in a photochemical reactor as defined in the first aspect of the disclosure herein above, and further described herein below.

The present disclosure, in a third aspect, typically pertains to a photo-oxidation processes and/or the photo-oxidation apparatus or photo-oxidation apparatus, respectively, wherein a light induced photochemical oxidation, typically an exothermic light induced photochemical oxidation, is performed in a photochemical reactor as defined in the first aspect of the disclosure herein above, and further described herein below, and which provide(s) for a chemically more comfortable and more environmentally friendly synthesis pathway for manufacturing active ingredients for pharma- and/or agro applications, for example, such active ingredients as referenced above, having a CF₃-group, CF₂-group, CCl₂-group or CCl₃-group, respectively, and to provide a building block required for the for manufacturing said active ingredients for pharma- and/or agro applications, for example, such active ingredients as referenced above, wherein in the building block C—F-bond(s) and/or C—Cl-bond(s) is (are) already present in the building block required as starting material to synthesize a before said active ingredient.

More typically, in this third aspect, the present disclosure pertains to a photo-oxidation processes and/or the photo-oxidation apparatus or photo-oxidation apparatus, respectively, wherein a light induced photochemical oxidation, typically an exothermic light induced photochemical oxidation, is performed in a photochemical reactor as defined in the first aspect of the disclosure herein above, and further described herein below, and which provide(s) for convenient starting materials building block(s), respectively, for direct usage or usage as a derivative thereof, for manufacturing active ingredients for pharma- and/or agro applications, for example, such active ingredients as referenced above, having a CF₃-group, CF₂-group, CCl₂-group or CCl₃-group, respectively, which are above said halogenated carboxylic acid chlorides, i.e., the polyfluorochloro- and/or perfluorocarboxylic acid chloride compounds.

Even more typically, in this third aspect, the present disclosure pertains to a photo-oxidation processes and/or the photo-oxidation apparatus or photo-oxidation apparatus, respectively, wherein a light induced photochemical oxidation, typically an exothermic light induced photochemical oxidation, is performed in a photochemical reactor as defined in the first aspect of the disclosure herein above, and further described herein below, and which provide(s) for convenient starting materials building block(s), respectively, for direct usage or usage as a derivative thereof, for manufacturing active ingredients for pharma- and/or agro applications, for example, such active ingredients as referenced above, having a CF₃-group, CF₂-group, CCl₂-group or CCl₃-group, respectively, which are halogenated carboxylic acid chlorides, i.e., the polyfluorochloro- and/or perfluorocarboxylic acid chloride compounds, are trifluoroacetyl chloride (TFAC), chlorodifluoroacetylchloride (CDFAC), trichloroacetylchloride (TCAC) and dichloroacetylchloride (DCAC).

In a forth aspect the present disclosure pertains to a novel photo-oxidation processes, in particular a more efficient and energy saving photo-oxidation processes and/or a more environmentally friendly photo-oxidation process, for the manufacture of organic compounds as defined herein above in the third aspect of the disclosure (i.e., a building block required for manufacturing active ingredients for pharma- and/or agro applications, for example, such active ingredients as referenced above, having a CF₃-group, CF₂-group, CCl₂-group or CCl₃-group, respectively) by photo-oxidation and/or by industrial scale photo-oxidation, and in particular by an exothermic photochemical reaction and/or industrial scale exothermic photo-oxidation, and wherein a light induced photochemical oxidation, typically an exothermic light induced photochemical oxidation, is performed in a photochemical microreactor, for example, in photochemical microreactor like the ones commercially available from Dow Corning (e.g., a type G1 photochemical microreactor; for example, equipped with 365 nm LEDs) or from Vapourtec (UK; e.g., a continuous flow photochemical microreactor, equipped with LED UV lightings), as further described herein below.

Reference is made to the FIGS. 6, 7 and 8, showing representatives of a photo-oxidation microreactor, used in the context of the present disclosure's photo-oxidation process, and fulfilling the inventive concept of interior installations forming a channel inside the reactor reservoir, for the manufacture of organic compounds as defined herein above in the third aspect of the disclosure (i.e., a building block required for manufacturing active ingredients for pharma- and/or agro applications, for example, such active ingredients as referenced above, having a CF₃-group, CF₂-group, CCl₂-group or CCl₃-group, respectively). The inventive concept of interior installations forming a channel inside the reactor reservoir, in the photochemical microreactor is represented, for example, by one or more interior installations (4^{M}) each forming a channel (6^{M}), i.e., which here is a microchannel (6^{M}), inside the reactor reservoir (2^{M}), i.e., which here is a microreactor reservoir (2^{M}), wherein the one or more interior installations (4^{M}) are in tube/pipe form and consist out of borosilicate glass, and wherein each of the interior installations (4^{M}) at both tube/pipe ends have an inlet/outlet installation (4^{M}a) and (4^{M}b) extending to the exterior of the reactor reservoir (2^{M}), i.e., which here is a microreactor reservoir (2^{M}).

The interior(s) of a photochemical reactor employed in context of the present disclosure is always made out of glass, which is chosen such that the wavelength of the UV lighting source (e.g., of an Hg-lamp or LED) required for inducing and maintaining the photochemical reaction can pass through the glass material(s). Specifically, in context of some aspects of the present disclosure the glass material typically is a borosilicate glass.

The interior installations are conceived by the present disclosure for heat dissipation and thus represent a common concept underlying the aspects of the present disclosure. Therefore, both all schemes (reactor variants) herein are equally described and backed up with examples to the improvements achieved over the related art, e.g., in terms of beneficial temperature management of photochemical reactions, and in particular of exothermic photochemical reactions. According to the present disclosure said temperature management is achieved by the interior installations as defined herein. For example, the temperature management is handled by interior installations such as tube(s)/pipe(s) or coil(s) in the photochemical reactor (e.g., see FIGS. 1 to 4, and 5A and 5B), which may be held in position by interior installations such as a multitude of gaskets (not shown in the drawings), or optionally in addition by interior installations such as a multitude of horizontal plates (5), e.g., baffle plate(s) (5) (e.g., see FIGS. 3, and 5A), or interior installations such as tube/pipe (e.g., microtube/micropipe) of a photochemical microreactor (e.g., see FIGS. 6 to 8). In principle, the tube(s)/pipe(s) are or coil(s) of the novel reactor designs of the disclosure, e.g., as shown by FIGS. 1, 2, 4, and 5B in the photochemical reactor, are comparable to the interior installations such as a multitude of horizontal plates (5), e.g., baffle plate(s) (5), e.g., as shown by FIGS. 3 and 5A, because due to the multiple deflection caused by the horizontal plates (5), e.g., baffle plate(s) (5), a flow of reaction medium is achieved by forming a channel that is mimicking a kind of the tube(s)/pipe(s) for the flow of the reaction medium therein, and according to disclosure in this case of novel reactor design the cooling liquid flows through the “regular” tube(s)/pipe(s), e.g., as shown in FIGS. 3 and 5A.

A similar principle, as shown e.g., as shown in FIGS. 3 and 5A, is implemented to practice in case of a coil reactor, e.g., as shown by FIGS. 4 and 5B. Herein, for example, a coil, e.g., an FEP coil, is forming tube(s)/pipe(s) around the UV lighting source, either (i) representing a channel through which the reaction medium flows whereas the cooling liquid cooling liquid flows through the reactor reservoir, or alternatively, (ii) the reactor reservoir is representing the a channel through which the reaction medium flows whereas the cooling liquid cooling liquid flows through tube(s)/pipe(s) arranged around the UV lighting source.

In case of a photochemical microreactor (e.g., as shown by FIGS. 6 to 8), according to the present disclosure it is conceived, like it is the case for the novel reactor designs of the disclosure (e.g., as shown by FIGS. 1 to 4, and 5A and 5B), a photochemical microreactor (photo-microreactor) contains all the necessary properties to control and manage the temperature of photochemical reactions, and in particular of exothermic photochemical reactions.

For example, a photochemical microreactor avoids hot spots due to excellent heat control and very good mixing of the reaction medium. However, in contrast to the novel reactor designs of the disclosure (e.g., as shown by FIGS. 1 to 4, and 5A and 5B), in a photochemical microreactor (e.g., as shown by FIGS. 6 to 8) only LEDs can be used as UV lighting source, and in current related art available LEDs yet are too weak to carry out industrial photochemistry; this minor restriction of used possibly will be resolved by development of more powerful LEDs in the future.

Nevertheless, the concept of the disclosure can actually also be practiced in a microreactor, even though due to the restricted lamp power (e.g., in Dow Corning's photochemical microreactor (photo-microreactor) yet no industrial production can be realized in large quantities (e.g., quantities of about ≥100 t). With the previously known photo microreactors, a maximum of 1 to 2 kg is probably the end.

Accordingly, in a photochemical microreactor the concept of the disclosure beneficially works on laboratory scale or even pilot scale, respectively, and in particular when applied for the manufacturing active ingredients for pharma- and/or agro applications, for example, such active ingredients as referenced above, having a CF₃-group, CF₂-group, CCl₂-group or CCl₃-group, respectively, and to provide a building block required for the for manufacturing said active ingredients for pharma- and/or agro applications, for example, such active ingredients as referenced above, wherein in the building block C—F-bond(s) and/or C—Cl-bond(s) is (are) already present in the building block required as starting material to synthesize a before said active ingredient.

In general, the advantage of the present disclosure, i.e., the use of a channel or a channels system, respectively, represented by tube(s)/pipe(s), coil(s) (e.g., coiled tube(s)/pipe(s)), which can be held in place by one or more gasket(s), and the optional horizontal plates (5), e.g., baffle plate(s) (5) installation(s) in combination with tube(s)/pipe(s), and interior installations such as tube/pipe (e.g., microtube/micropipe) of a photochemical microreactor, is achieved in that by each of said interior installations avoid formation of hot spots, and important provide for very good mixing of the reaction medium such that the photochemical reactions do not only take place in the part of reaction medium which a few mm around the UV lighting source. Accordingly, by the concept of the present disclosure, e.g., by the inventive interior installations in a photo reactor or by use of a photochemical microreactor, respectively, as often as possible, the starting material must be forced to pass alternately through the UV light exposed zone (reaction zone in narrower sense) around the UV lamp (UV lighting source) and also the heat exchanger zone or cooling zone, respectively, in the photo reactor. Accordingly, the reaction medium is to pass alternately subjected to UV light exposure and to cooling.

The aspects the present disclosure shall be described in more detail herein after. The detailed description of the disclosure and the subject-matter of the claims firstly are directed to the inventive aspects of the apparatus for photo-oxidation, i.e., the aspects and feature of the novel photochemical reactor; secondly on the photochemical process performed therein, in particular the two variants of type a) and b) of photo-oxidation; and then thirdly to the “chemistry” of photo-oxidation, in particular the manufacturing of building blocks for active ingredients for pharma- and/or agro applications, for example, such active ingredients as referenced above, having a CF₃-group, CF₂-group, CCl₂-group or CCl₃-group, respectively, which building blocks in a preferred aspect of the disclosure, e.g., are above said halogenated carboxylic acid chlorides, i.e., the polyfluorochloro- and/or perfluorocarboxylic acid chloride compounds.

As already pointed out further above, although photo-oxidation reactions are very clean-type light induced reactions with high yields and high selectivity, agent molecular oxygen may cause some disadvantages in photochemical oxidations especially such ones performed at lower temperatures. The reason is the potential formation of undesired “oxidizing material” or “oxidizing by-products,” respectively. Without wished to be bound by theory, it is assumed that the formation of said oxidizing material or oxidizing by-products is caused by the activeness of oxygen (e.g., light induced increased reactivity of oxygen, “activated oxygen”) and subsequently of peroxides during the photochemical oxidation. For example, such undesired oxidizing material or oxidizing by-products can be found in a photochemical reactor system and possibly accumulate at certain parts and/or certain positions of photochemical reactor system and/or process equipment used in context of a photochemical oxidation, especially at positions where some potential condensation can occur.

For example, such positions at which such condensation of oxidizing material or oxidizing by-products, e.g., presumably peroxide compounds formed out of the starting material(s), can occur can be found in cooling traps or in a pilot plant or industrial unit at positions of the piping system, especially because such potential condensation is depending on dew point of the entire photochemical reaction mixture. Said oxidizing material or oxidizing by-products, respectively, is found to be very corrosive. In the contrary, in context of the present disclosure it was found that the formed acid chlorides are non-corrosive so that, e.g., trifluoroacetyl chloride (TFAC) can be handled in reactors and or equipment made out of or surface-covered with carbon black steel.

In this aspect of the present disclosure the decomposition of the suspected peroxides (e.g., the assumed oxidizing material or oxidizing by-products) is proposed by the present disclosure, especially as such decomposition is regarded necessary under safety aspects, and also to avoid corrosion in the downstream parts of the apparatus, but in addition the decomposition process proposed in this aspect of the present disclosure is found to increase yield of the desired photochemical oxidation products as well.

The chemical structure(s) of above said oxidizing material and/or oxidizing by-products could not be confirmed by related art analytical methods. However, based on the findings of the present disclosure, for example, for the reason that contact(s) thereof with initiating material(s) resulted in the formation of acid chlorides, and in view of the fact, e.g., that trifluoroacetyl chloride (TFAC) is a gaseous product at room temperature leads to the conclusion that by treating said oxidizing material and/or oxidizing by-products (e.g., presumably being peroxides) with initiating material(s) such like, for example, carbon black steel, surprisingly provides a simple and effective method or process for eliminating said oxidizing material and/or oxidizing by-products from a reaction mixture obtained from a photochemical oxidation.

For illustration, but without being bound to a particular theory, in the scheme herein after potential reaction mechanism for non-sensitized photo-oxidation and for chlorine sensitized photo-oxidation are displayed, as exemplified for TFAC. In the case of the photo-oxidations for manufacturing other polyfluorochloro- and/or perfluorocarboxylic acid chloride compounds (than TFAC), the potential reaction mechanism for non-sensitized photo-oxidation and for chlorine sensitized photo-oxidation is deemed analogous for the appropriate starting material, and corresponding products are formed, accordingly.

### Potential Reaction Mechanism for Non-Sensitized Photo-Oxidation (Example TFAC); the Desired “Main” Reaction is Shown, as Well as the Undesired Peroxide Formation in the “Side” Reaction Pathway::

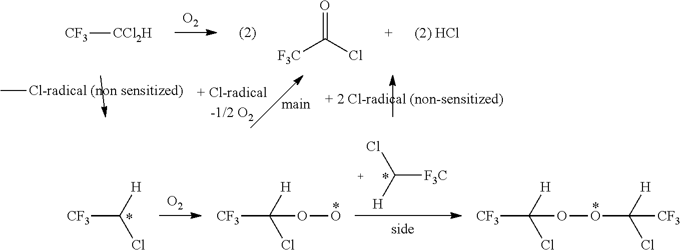

### Potential Reaction Mechanism for Chlorine Sensitized Photo-Oxidation (Example TFAC):

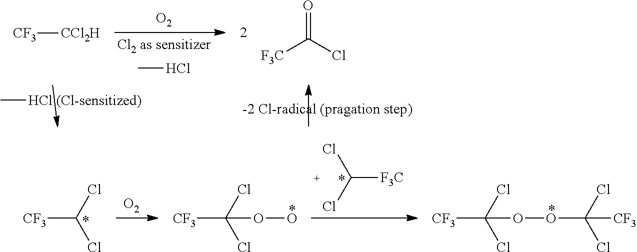

The structure of trifluoroacetyl chloride (TFAC) formed after treatment of said oxidizing material and/or oxidizing by-products with initiating materials was confirmed by GC-MS (combined Gas-Chromatography/Mass-Spectroscopy). Apart from confirming its presence by collecting it as a liquid in gas-phase process, said oxidizing material and/or oxidizing by-products were also measured and tracked by DSC (differential scanning calorimetry).

Without being bound to a particular theory, it is assumed that in the course of the reaction according to the mechanism shown in the scheme above, i.e., in the course of the potential reaction mechanism for non-sensitized photo-oxidation and for chlorine sensitized photo-oxidation displayed above, organic peroxide side-products are formed. In chemistry, peroxides are understood as a group of compounds with the structure R—O—O—R′, where R and R′, independently can be any element. The O—O group in the peroxide is called the peroxide group or peroxo group. Organic peroxides are organic compounds with the structure R—O—O—R′ containing the peroxide functional group O—O, and wherein R and R′, independently at least one thereof means an organic substituent. Accordingly, for example, in view of said potential reaction mechanism, as exemplified for TFAC, it is assumed that the following organic peroxide side-products are formed:

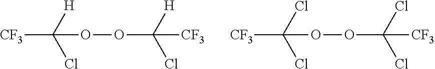 [SMILES: FC(F)(F)C(Cl)OOC(Cl)C(F)(F)F ; FC(F)(F)C(Cl)(Cl)OOC(Cl)(Cl)C(F)(F)F]

In the case of the photo-oxidations for manufacturing other polyfluorochloro- and/or perfluorocarboxylic acid chloride compounds (than TFAC), in view of analogous potential reaction mechanism, from the appropriate starting material, and corresponding products are formed, and it is assumed as well that corresponding organic peroxide side-products are formed, accordingly.

As already stated above, said oxidizing material and/or oxidizing by-product(s) are deemed to be organic peroxide(s) and being formed as by-product(s) or side-product(s), respectively, in the photochemical oxidation, according to the findings of the present disclosure these can by decomposed by converting them into the desired acid product(s), i.e., the respective acid chloride(s), by contacting said oxidizing material(s) and/or oxidizing by-product(s), e.g., the presumed organic peroxide(s) formed from the respective starting material(s), with “initiating material(s)” such like, for example, carbon black steel. Surprisingly, said contacting with “initiating material(s)” provides a simple and effective method or process for eliminating said oxidizing material(s) and/or oxidizing by-product(s) from a reaction mixture obtained from a photochemical oxidation.

In chemistry, the term “initiation” means a chemical reaction that triggers one or more secondary reactions. Often the initiation reaction generates a reactive intermediate from a stable molecule which is then involved in secondary reactions.

Accordingly, the terms “initiation” or “initiator” or “initiating material(s)” and the like terms, and as applicable in the context of the present disclosure are to be understood in context of a potential reaction mechanism involving material (s capable of initiating the formation of radicals, e.g., of free radicals. In chemistry, the term “radical” or “free radical” means an atom, molecule, or ion that has at least one unpaired valence electron. With some exceptions, these unpaired electrons make radicals highly chemically reactive and most organic radicals have only short lifetimes. Radicals may be generated or initiated in a number of ways, but typical methods involve redox reactions.

Accordingly, for example, in view of said potential reaction mechanism, as exemplified for TFAC, it is assumed that by contacting the above shown organic peroxide side-products with radical “initiating material(s)” the following organic oxo-radicals are formed, and then are further reacting to the desired acid product, i.e., in this example case to TFAC.

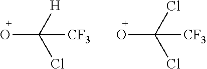 [SMILES: OC(Cl)C(F)(F)F ; OC(Cl)(Cl)C(F)(F)F]

In the case of the photo-oxidations for manufacturing other polyfluorochloro- and/or perfluorocarboxylic acid chloride compounds (than TFAC), in view of analogous potential reaction mechanism, from the appropriate starting material, and the corresponding products formed, and it is assumed as well that corresponding oxo-radicals are formed, accordingly, and then are further reacting to the desired corresponding acid products, e.g., the desired acid chlorides.

Accordingly, the terms “initiation” or “initiator” or “initiating material(s)” and the like terms, and as applicable in the context of the present disclosure, mean a material that in contact with an organic peroxide generates and/or initiates the formation of the corresponding organic oxo-radicals of said organic peroxide.

Particular examples of such initiating material(s) are: carbon black steel or carbon steel; corroded metals (e.g., corroded steel); transition metal oxides wherein the metal is selected from the group consisting of iron, cobalt, copper (e.g, copper oxide, CuO), nickel and zinc; transition metal chlorides wherein the metal is selected from the group consisting of iron, cobalt, copper, nickel (e.g., nickel chloride, NiCl₂), tungsten and zinc; and also including mixtures of said oxides, chlorides and/or mixtures of said oxides and chlorides; and carbon, e.g. including activated carbon, deactivated active carbon or pre-treated active carbon, e.g., as described hereunder.

Active carbon obtained directly from its production has very high surface area and thereby is such reactive that, for example, any oxidizing material contacting an area of the active carbon could decompose immediately and extremely fast at the initial contact area, e.g., like the reactor and/or pipe entrance areas (hot spot formation), very likely also increasing the risk of such high heat that in a worst case some unreacted oxygen together with the active carbon could get burning. Thus, the active carbon typically is slowly pre-loaded with starting material (e.g., with HCFC-123 in case of making TFAC) or with product material (e.g., with TFAC in case of making TFAC from HCFC-123) in order to reduce the reactivity of the active carbon, and to allow for more slowly and more controlled decomposing any oxidizing material. Without deactivation or pre-treatment, respectively, of said directly obtained active carbon, the gas flow (flow gaseous starting material) in the photochemical reaction would get so hot that said gas flow, as mentioned here before, could even start burning with oxygen still present in case of incomplete conversion, and/or could lead to tars and/or other undesired by-products. Thus, for example, in the case of photo-oxidation for manufacturing TFAC, pre-treatment of active carbon or pre-treated active carbon means that prior to the photo-oxidation the activated carbon is slowly dosed dropwise or slowly sprinkled, respectively, with the liquid starting material HCFC-123 so that the active carbon finally even stands in the liquid starting material, and then such pre-treated active carbon is filtered off. In the case of the photo-oxidations for manufacturing other polyfluorochloro- and/or perfluorocarboxylic acid chloride compounds (than TFAC), the appropriate starting material therefore is used for the pre-treatment of active carbon is used accordingly.

Accordingly, in general the terms “deactivating,” “pre-treating” of active carbon, “deactivated active carbon” or “pre-treated active carbon” and the like terms, and as applicable in the context of the present disclosure, means that active or activated carbon, e.g., freshly prepared active carbon and/or active carbon, in particular such active carbon obtained directly from its production, prior to the photo-oxidation is slowly dosed dropwise or slowly sprinkled, respectively, with the liquid starting material for manufacturing a polyfluorochloro- and/or perfluorocarboxylic acid chloride compound by photo-oxidation so that the active carbon finally even stands in the liquid starting material, and then such deactivated or pre-treated active carbon is filtered off. The term “slowly” in context of slowly dosing dropwise or slowly sprinkling means that starting material for manufacturing a polyfluorochloro- and/or perfluorocarboxylic acid chloride compound by photo-oxidation is dosed dropwise or sprinkled such that the self-heating of the active or activated carbon is confined at maximum to the boiling point of the respective starting material that is dosed dropwise or sprinkled thereupon.

The initiating material, as described here before, used to decompose any oxidizing material can be applied (batch-wise) after having collected and separated off said oxidizing material from the desired product stream. However, collecting the oxidizing material, may involve a certain risk potential due to the accumulation, in particular in case of peroxides formed as the oxidizing material. Accordingly, in a preferred process variant of the photochemical reaction of the present disclosure, more typically in a pilot plant or industrial plant scale of the photochemical oxidation process, oxidizing material is not separated off from the desired product stream and said oxidizing material is not collected, but the initiating material, as described here before, used to decompose any oxidizing material can be applied is applied continuously any oxidizing material. Such continuous decomposition of any oxidizing material (e.g., of peroxides) can be achieved in that a container or a pipe filled with initiating material (e.g., filled with carbon, e.g. including activated carbon, deactivated active carbon or pre-treated active carbon) is installed after the photochemical reactor, and the product stream, which possibly is comprising any oxidizing material, flows through said container or pipe which is filled with initiating material (i.e., the “decomposition container” or “decomposition pipe”), before the product stream leaving said decomposition container or decomposition pipe is further processed to a work-up. In case of a gas product stream leaving said decomposition container or decomposition pipe, then for working-up the gaseous product stream normally goes into a compressor that feeds a pressure distillation, thus after distillation yielding the final product, which is gaseous at room temperature and atmospheric pressure). In case of a final product which is liquid at room temperature and atmospheric pressure (a liquid product) but a gas mixture at reaction conditions, the reaction mixture passes said decomposition container or decomposition pipe in gas phase, but, then for working-up the condensed and then liquid product stream normally goes directly into a distillation (without any compression) even under applying some vacuum if necessary.

The continuous decomposition procedure is further exemplified, for the manufacture of TFAC as described further above, and reference is made to the reaction scheme displayed in FIG. 9. As a guideline, the size (volume) of the decomposition reactor (for the decomposition of the peroxides) is 1/10 in relation to the reactor volume for the photo-oxidation. Reference is made to Example 21 of the disclosure.

### Definitions

Some further definitions of terms used herein are given here below, in addition to those terms already defined in the Summary of the Disclosure.

The terms “vertical,” “horizontal” or “vertical direction,” “horizontal direction” and the like, if not expressively defined otherwise herein, in context of the present disclosure mean the relative arrangement of interior installation(s) in a photochemical reactor in relation to the bottom surface of a reactor reservoir wherein said interior installation(s) are arranged. Thus, in this regard the terms “vertical” or “vertical direction,” each in relation to the bottom surface of a reactor reservoir, synonymously mean positioning “upright” or in “upright direction,” or positioning “perpendicular” or in “perpendicular direction,” respectively. Thus, in this regard the terms “horizontal” or “horizontal direction,” each in relation to the bottom surface of a reactor reservoir, synonymously mean positioning “parallel” or in “parallel direction,” respectively.

The term “carbon black steel” or “carbon steel” in context of the present disclosure means steel with carbon content from about 0.05 up to 2.1 percent by weight. The definition of carbon steel from the American Iron and Steel Institute (AISI) states further constituents that may be comprised as follows: no minimum content is specified or required for chromium, cobalt, molybdenum, nickel, niobium, titanium, tungsten, vanadium, zirconium, or any other element to be added to obtain a desired alloying effect; the specified minimum for copper does not exceed 0.40 percent by weight; or the maximum content specified for any of the following elements does not exceed the percentages noted: manganese 1.65 percent by weight; silicon 0.60 percent by weight; copper 0.60 percent by weight. The term “carbon steel” may also be used in reference to steel which is not stainless steel; in this use carbon steel may include alloy steels.

The term “room temperature” and the like terms, and as applicable in the context of the present disclosure, is usually understood by a person skilled in chemical art as a temperature in the range of 20° C. to 25° C.

The numerical ranges disclosed herein include all values from, and including, the lower and upper value. For ranges containing explicit values (e.g., 1 to 7), any subrange between any two explicit values is included (e.g., 1 to 2; 2 to 6; 5 to 7; 3 to 7; 5 to 6; etc.).

The term “photochemistry”: Photochemistry is the branch of chemistry, well-known to the person skilled in the art, concerned with the chemical effects of light. Generally, this term is used to describe a chemical reaction caused by absorption of ultraviolet light (abbreviated “UV;” wavelength from 100 to 400 nm), visible light (400 to 750 nm), or infrared radiation (750 to 2500 nm). In context of the present disclosure UV is used as the lighting source(s) and for design of photo reactor(s), and for performing the photochemical oxidation(s) of the disclosure.

The term “photon(s)”: Photochemistry makes use of the energy of photon(s), here in context of the disclosure of photon(s) emitted by a UV lighting source. In quantum physics, light is no longer understood as a classical wave, but as a quantum object. Accordingly, light is made up of individual discrete energy quanta, the so-called photons. A photon is an elementary particle, more precisely an elementary boson with a mass of 0, which always moves with the speed of light “c.” It has an energy of E=hv. Therein “v” is the frequency of the light and “h” is Planck's constant with h=6.62607015*10 34 Js. The photon has a momentum of p=h/λ, wherein “λ” is the wavelength of the light.

The term “wavelengths”: In physics, as well known to the person skilled in the art, generally the wavelength is the spatial period of a periodic wave, i.e., the distance over which the wave's shape repeats. It is the distance between consecutive corresponding points of the same phase on the wave, such as two adjacent crests, troughs, or zero crossings, and is a characteristic of both, traveling waves and standing waves, as well as other spatial wave patterns. The inverse of the wavelength is called the spatial frequency. Wavelength is commonly designated by the Greek letter lambda (λ). Assuming a sinusoidal wave moving at a fixed wave speed, wavelength is inversely proportional to frequency of the wave: waves with higher frequencies have shorter wavelengths, and lower frequencies have longer wavelengths. Wavelength depends on the medium (for example, vacuum, air, or water) that a wave travels through. In the context of the present disclosure light, i.e., such as UV-light, is an example of a wave. In light, i.e., such as UV-light, and other electromagnetic radiation the strength of the electric and the magnetic field vary. The range of wavelengths or frequencies for wave phenomena is called a spectrum.

The term “ultraviolet (UV)”: Ultraviolet (UV) is a form of electromagnetic radiation, well-known to the person skilled in the art, with wavelength as indicated herein before, shorter than that of visible light, but longer than X-rays. UV radiation can be produced by electric arcs and specialized lights, such as mercury-vapor lamps, tanning lamps, and black lights. Although long-wavelength ultraviolet is not considered an ionizing radiation because its photons lack the energy to ionize atoms, it can cause chemical reactions and causes many substances to glow or fluoresce. Consequently, the chemical and biological effects of UV are greater than simple heating effects, and many practical applications of UV radiation derive from its interactions with organic molecules.

Accordingly, ultraviolet radiation, or UV radiation, respectively, is normally understood by the person skilled in the art as the wavelength range in the electromagnetic spectrum that immediately follows the short-term end of the visible range, the violet. The International Commission d'Éclairage (ICE) defines UV as the wavelength range between 400 nm (limit to visible light) and 100 nm (start of the range of X-rays); that is the wavelength range in its broadest scope concerned also by the present disclosure.

Concerning the effects and applications of UV radiation it well known to the person skilled in the art that UV radiation is more energetic than visible light. Their energy is sufficient to destroy chemical bonds or to generate reaction partners and enable them to form new bonds. Since chemical bonds have different strengths, specific bonds can be selectively stimulated or broken by UV light of different energies or wavelengths and thus different chemical processes can be controlled.

Also, it is well known to the person skilled in the art that different areas of application necessitate specific requirements on UV components and UV systems, since not only the wavelength of the radiation is decisive, but also, depending on the area of application, certain minimum irradiance levels or radiation levels (dose) must be achieved. Furthermore, areas of different sizes, including areas that are very large in relation to the radiation source, often have to be illuminated homogeneously. In this regard, the person skilled in the art will be capable to make modifications and/or adaptations as deemed necessary for a concerned photochemical reaction to be performed, e.g., also in respect of photochemical oxidation of the present disclosure.

The electromagnetic spectrum of ultraviolet radiation (UVR), defined most broadly as 100 to 400 nm (nanometers), in context of the present disclosure and in line with recommendation by standards, e.g., by the ISO standard ISO-21348, can be subdivided into a number of ranges, and applicable to the present disclosure as follows:

| | | | Photo |
|---|---|---|---|
| Name | | Wavelength | Energy |
| (Alternative Name) | Abbreviation | (nm) | (eV, aJ) |
| Ultraviolet A | UV-A | >315-400 | 3.10-3.94, |
| (long-wave UV, black | | | 0.497-0.631 |
| light, soft UV) | | | |
| Ultraviolet B | UV-B | >280-315 | 3.94-4.43, |
| (medium-wave UV, | | | 0.631-0.710 |
| intermediate UV; | | | |
| Dorno radiation) | | | |
| Ultraviolet C | UV-C | 100-280 | 4.43-12.4, |
| (Short-wave UV, | | | 0.710-1.987 |
| germicidal UV, ionizing | | | |
| radiation at shorter | | | |
| wavelengths, hard UV) | | | |

The term “mercury-vapor lamp” or “Hg-lamp,” respectively, is well known to the person skilled in the art. A mercury-vapor lamp is a gas-discharge lamp that uses an electric arc through vaporized mercury to produce light, i.e., such as UV-light. The arc discharge is generally confined to a small fused quartz arc tube mounted within a larger borosilicate glass bulb. The outer bulb may be clear or coated with a phosphor; in either case, the outer bulb provides thermal insulation, protection from the ultraviolet radiation the light produces, and a convenient mounting for the fused quartz arc tube. Mercury vapor lamps are more energy efficient than incandescent and most fluorescent lights, with luminous efficacies of 35 to 65 lumens/watt. As an example, some information on the emission line spectrum is given here in terms of wavelengths (nm): 184.45 nm, ultraviolet (UVC); 253.7 nm, ultraviolet (UVC); 365.4 nm, so-called “I-line,” ultraviolet (UVA); 404.7 nm, so-called “H-line,” violet; 435.8 nm, so-called “G-line,” blue. In low-pressure mercury-vapor lamps only the lines at 184 nm and 254 nm are present. Fused silica is used in the manufacturing to keep the 184 nm light from being absorbed. In medium-pressure mercury-vapor lamps, the lines from 200 to 600 nm are present. The lamps can be constructed to emit primarily in the UV-A (around 400 nm) or UV-C (around 250 nm).

The term “Light-emitting diodes” or “LEDs,” respectively, is well known to the person skilled in the art. Light-emitting diodes (LEDs) are semiconductors that convert electrical energy into light energy. The color of the emitted light depends on the semiconductor material and composition, with LEDs generally classified into three wavelengths: ultraviolet, visible, and infrared. The wavelength range of commercially available LEDs with single-element output power of at least 5 mW is 275 to 950 nm. Each wavelength range is made from a specific semiconductor material family, regardless of the manufacturer. The wavelength range of commercially available ultraviolet LEDs (UV LEDs) is normally 240 to 360 nm. UV LEDs are specifically used for a variety of industrial applications, and can be also applied in context of the present disclosure, i.e, in context of phot-oxidation. Power output levels greater than 100 mW can been achieved at wavelengths as short as 280 nm. The material primarily used for UV LEDs is gallium nitride/aluminum gallium nitride (GaN/AlGaN) at wavelengths 360 nm or longer. Shorter wavelengths utilize other commercially available (but possibly proprietary) materials. There is a stable supply market for wavelengths 360 nm and longer, at lower prices and plentiful suppliers. Shorter wavelengths are also commercially available, but at present manufactured by only a few suppliers. Regarding near-UV to green LEDs the wavelength range of commercially available LEDs is normally 395 to 530 nm. The material for this wavelength range of products is indium gallium nitride (InGaN). It is technically possible to make a wavelength anywhere between 395 and 530 nm.

Light-emitting diodes (LEDs) can be manufactured to emit radiation in the ultraviolet range. In 2019, following significant advances over the preceding five years, UV-A LEDs of 365 nm and longer wavelength were available, with efficiencies of 50% at 1.0 W output. Currently, the most common types of UV LEDs that can be found/purchased are in 395 nm and 365 nm wavelengths, both of which are in the UV-A spectrum. When referring to the wavelength of the UV LEDs, the rated wavelength is the peak wavelength that the LEDs put out, and light at both higher and lower wavelength frequencies near the peak wavelength are present, which is important to consider when looking them to apply for certain purposes. The currently more common 395 nm UV LEDs are much closer to the visible spectrum, and LEDs not only operate at their peak wavelength, but they also give off a purple color, and end up not emitting pure UV light, unlike other UV LEDs that are deeper into the spectrum. UV-C LEDs are also developing rapidly, but may require testing to verify their effectiveness for the purpose of use.

The term “gasket” or here “tube/piping gasket,” respectively, and similar terms, denotes a mechanical seal which fills the space between two or more mating surfaces, generally to prevent leakage from or into the joined objects while under compression. Given the potential cost and safety implications of faulty or leaking gaskets, it is critical that the correct gasket material is selected to fit the needs of the application. A gasket is a deformable material that is used to create a static seal and maintain that seal under various operating conditions in an assembly. Construction, material and use of “gasket” or here “tube/piping gasket,” respectively, is known to the person skilled in the art, for many technical purposes and conditions. The gaskets can be made out of fluorinated polymers like FFKM (Kalrez) or HDPTFE (high density polytetrafluoroethylene). Fluorinated polymers like those derived from PE (polyethylene) and PET (polyethylene terephthalate or poly(ethylene terephthalate) are less suitable, as PE and PET will swell in contact with the reaction medium and loose sealing properties after short time. Here in context of the disclosure, gaskets are normally made from a flat material, for example, a sheet such as polytetrafluoroethylene (otherwise known as PTFE or Teflon) or a halogenated plastic polymer such as polychlorotrifluoroethylene.

The term “baffle,” “baffle plate,” “turning plate,” “deflecting plate,” “deflector plate,” “plate baffle” sometimes also simply called “plate,” and similar terms, denotes an installation device known in the technique as flow-directing or obstructing vanes or panels used to direct a flow of liquid or gas. It is used in some household stoves and in some industrial process vessels (tanks), such as shell and tube heat exchangers, chemical reactors, and static mixers. Baffles, for example, are an integral part of the shell and tube heat exchanger design. A baffle is designed to support tube bundles and direct the flow of fluids for maximum efficiency.

The term “photochemical microreactor,” and similar terms, denotes commercially available microreactors in general for performing photochemical reactions, especially in context of the disclosure for performing photochemical oxidations, equipped with UV lightings (e.g. UV light emitting LEDs), e.g., in particular also such wherein UV lightings (e.g. UV light emitting LEDs) are integrated to glass channels (having micrometer dimension), e.g., thus forming an UV (e.g. UV light emitting LEDs) irradiated glass channels (having micrometer dimension).

The term “borosilicate glass,” and similar terms, denotes commercially available type of glass with silica (silicon dioxide; SiO₂) and boron trioxide as the main glass-forming constituents. Borosilicate glasses are known for having very low coefficients of thermal expansion (≈3×10−6 K−1 at 20° C.), making them more resistant to thermal shock than any other common glass. Such glass is subjected to less thermal stress and can withstand temperature differentials without fracturing of about 165° C. (297° F.). It is commonly used for the construction of reagent bottles and flasks as well as lighting, electronics and cookware. Borosilicate glass is sold under various trade names, including Borosil®, Duran®, Pyrex®, Supertek®, Suprax®, Simax®, Bellco®, Marinex® (Brazil), BSA® 60, BSC® 51 (by NIPRO), Heatex®, Endural®, Schott®, Refmex®, Kimax®, Gemstone Well®, and MG® (India). For example, Duran® glass is a brand name for the internationally defined borosilicate glass 3.3 (DIN ISO 3585). Because of its high resistance to heat and temperature changes, as well as its high mechanical strength and low coefficient of thermal expansion, Duran®, which Pyrex® from Corning is similar to, is widely used for laboratory devices, but also in cathode ray tubes, transmitting tubes, and speculums, and is case for the present disclosure also for UV lightings. Borosilicate glass, e.g., such as Duran® or Pyrex®, have advantageous properties such as, for example, high chemical resistance, outstanding transmission properties, high thermal capacity and resistance to thermal shock, is a strong electrical insulator, has transparency, high resistance to scratches, and has easily cleaned smooth surface.

The term “quartz” or “quartz glass,” and similar terms, denotes commercially available type of glass made out of the crystalline mineral known as quartz. Quartz is a hard, crystalline mineral composed of silica (silicon dioxide). Silicon dioxide, also known as silica, is an oxide of silicon with the chemical formula SiO₂, most commonly found in nature as quartz. The atoms are linked in a continuous framework of SiO₄ silicon-oxygen tetrahedrons, with each of the oxygens being shared by two tetrahedrons, giving an overall chemical formula of SiO₂. Quartz is the second most abundant mineral. Quartz exists in two forms, the normal α-quartz and the high-temperature β-quartz, both of which are chiral. The transformation from α-quartz to β-quartz takes place abruptly at 573° C. (846 K; 1,063° F.).

The term “PTFE,” and similar terms, denotes commercially available fluorinated ethylene polymer. Polytetrafluoroethylene (PTFE) is a synthetic fluoropolymer of tetrafluoroethylene that has numerous applications. The commonly known brand name of PTFE-based compositions is Teflon by Chemours, Polytetrafluoroethylene is a fluorocarbon solid (at room temperature), as it is a high-molecular-weight polymer consisting wholly of carbon and fluorine. PTFE is hydrophobic: neither water nor water-containing substances wet PTFE. Polytetrafluoroethylene is non-reactive, partly because of the strength of carbon-fluorine bonds, and so it is often used in containers and pipework for reactive and corrosive chemicals.

The term “HDPTFE,” and similar terms, denotes commercially available high density polytetrafluoroethylene.

The term “FKM,” and similar terms, denotes commercially available fluorocarbon-based fluoroelastomer materials. Hence, FKM is a family of fluorocarbon-based fluoroelastomer materials defined by the ASTM International standard D1418, while it is called FPM by ISO 1629. It is commonly called fluorine rubber or fluoro-rubber. All FKMs contain vinylidene fluoride as a monomer. FKMs are today produced by many companies, including: Chemours (Viton®), Daikin (Dai-El®), 3M (Dyneon®), Solvay S.A. (Tecnoflon®), HaloPolymer (Elaftor®), Gujarat Fluorochemicals (Fluonox®), Zrunek (ZruElast®), and several Chinese manufacturers including VSK Industrial. Fluoroelastomers provide additional heat and chemical resistance.

The term “FFKM,” and similar terms, denotes commercially available fluorocarbon-based fluoroelastomer materials related to “FKM.” FFKMs (by ASTM 1418 standard) (equivalent to FFPMs by ISO/DIN 1629 standard) are perfluoroelastomeric compounds, e.g. such as Kalrez®, containing an even higher amount of fluorine than FKM fluoroelastomers. They have improved resistance to high temperatures and chemicals and even withstand environments where oxygen-plasma are present for many hours. Certain grades have a maximum continuous service temperature of 327° C. (621° F.). They are commonly used to make O-rings and gaskets that are used in applications that involve contact with hydrocarbons or highly corrosive fluids, or when a wide range of temperatures is encountered.

The term “FEP,” and similar terms, denotes commercially available fluorinated ethylene propylene polymer. Fluorinated ethylene propylene (FEP) is a copolymer of hexafluoropropylene and tetrafluoroethylene. It differs from the polytetrafluoroethylene (PTFE) resins in that it is melt-processable using conventional injection molding and screw extrusion techniques. Fluorinated ethylene propylene (FEP) was invented by DuPont and is sold under the brandname Teflon FEP. Other brandnames are Neoflon FEP from Daikin or Dyneon FEP from Dyneon/3M. FEP is very similar in composition to the fluoropolymers PTFE (polytetrafluoroethylene) and PFA (perfluoroalkoxy polymer resin). FEP and PFA both share PTFE's useful properties of low friction and non-reactivity, but are more easily formable. FEP is softer than PTFE and melts at 260° C.; it is highly transparent.

The term “plastic material” or “polymeric material,” respectively, and similar terms, as used here in the context of the disclosure, denotes a fluorinated polymeric material which is transparent for a UV wavelength as applied in a photochemical reaction according to the disclosure, typically as applied in a photo-oxidation, and which fluorinated polymeric material is resistant to the reaction conditions. Particular examples of such “plastic material” or fluorinated polymeric material, respectively, are the fluorinated polymeric materials as mentioned herein above, for example: PTFE, HDPTFE, FKM, FFKM, and/or FEP.

In context of the present disclosure, as summarized above in the Summary of the Disclosure, now it was found, for example, that in small-scale with laboratory equipment (e.g., with a reactor length of about 30 cm) quite good to at least acceptable yields (and quite good to at least acceptable selectivity could be obtained. For example, photo-oxidation of PER to TCAC gives yields of about 80%, photo-oxidation of HCFC-123 to TFAC gives yields of about 85%, photo-oxidation of HCFC-122 to CDFAC gives yields of about 65%. But in a pilot plant and production plant where the reactor length is about 10 times longer (e.g., a length of about 2 to 3 m or even more) as compared to a laboratory equipment, a very high exothermic activity and formation of local “hot spots” in the photo reactor leads to a significant reduction in the yields and in the selectivity. These problems or disadvantages of the related art photo-oxidation are resolved by the current disclosure by a “special” design of a photochemical reactor, e.g., internal installations in the photochemical reactor forming channel(s) as defined herein and as further described herein below, such that the reaction media is permanently either forced by said “special” design to pass over internal or external heat exchangers (horizontal push) and/or to circulate in one or more channels inside the photochemical reactor reservoir along or around a UV lighting source of a photochemical reactor, e.g., along or around an Hg-lamp or LED UV lighting. The one or more channels (6) inside the photochemical reactor reservoir can be construed by one or more interior installations (4) which are in tube/pipe, or also by interior installations (5) inside the reactor reservoir (2), wherein the two or more interior installations (5) divide the reactor reservoir (2) into a further channel system (6a), Furthermore, the one or more channels (6) inside the photochemical reactor reservoir can be construed by one or more interior installations (4) which are in the form of a coil, e.g. a glass or plastic coil, arranged around a UV lighting source (which can be an Hg-lamp or LED-lamp, or even a laser-lamp) of a photochemical reactor; or the one or more channels (6) inside the photochemical reactor reservoir can be construed as a microchannel of a photochemical microreactor. Here it is readily understood by the person skilled in the art of photochemical oxidation the material(s), e.g. a glass or, if technically suitable, also plastic material, used for making the one or more interior installations of the photochemical reactor or photochemical microreactor, respectively, are chosen such that the wave length of the UV lighting source (e.g., of an Hg-lamp or LED) required for inducing and maintaining the photochemical reaction can pass through the material(s), e.g. through a glass material or, if technically suitable, also, plastic material.

Regarding the materials mentioned here before, e.g., glass material or, if technically suitable, also, plastic material, the following is noted in context with the present disclosure. The surroundings of a mercury lamp (Hg-lamp), which can reach a temperature of up to 800° C., must of course always be made of glass. In case of other UV lighting sources, e.g., LED UV lighting source, plastic material is an alternative option, but glass material can be used also together with LED UV lighting source. The reaction material(s) can of course flow through glass or plastic parts of the photo reactor, under the condition said before, and importantly under the condition that the photo reactor material glass or plastic is transparent for the UV wavelength required for the concerned photochemical reaction, e.g., for the photochemical oxidation.

Regarding the working temperature of the reaction mixture, according to the present disclosure, it is noted that the temperature is chosen such that all components of the reaction medium still remain in the gas phase, since the chemistry of photo-oxidation according to the present disclosure takes place in the gas phase. For example, when manufacturing TFAC by photo-oxidation according to the present disclosure the reactor temperature is set, e.g., at about 40° C.; the boiling point of TFAC is −27° C., and thus the reaction temperature will be set in any case at a temperature of about sufficiently above −27° C. to keep TFAC in the gas phase. For example, when manufacturing CDFAC by photo-oxidation according to the present disclosure the reactor temperature is set at about 70° C., e.g., at a higher temperature than that for manufacturing TFAC; the boiling point of CDFAC is 20° C., and thus the reaction temperature will be set in any case at a temperature of about sufficiently above 20° C. to keep CDFAC in the gas phase. The boiling point of TCAC is 117.9° C., and accordingly, for example, when manufacturing TCAC the reaction temperature will be set at a temperature of about sufficiently above 117.9° C. to keep TCAC in the gas phase. The boiling point of DCAC is 107° C., and accordingly, for example, when manufacturing DCAC the reaction temperature will be set at a temperature of about sufficiently above 107° C. to keep DCAC in the gas phase.

If a so called photochemical microreactor like the ones (type G1) from Dow Corning, e.g., with 365 nm LEDs as UV lighting source, or continuous flow photochemical microreactors from Vapourtec (UK) or any other photochemical reactor equipped with LED UV lighting, respectively, shall be employed for performing a photochemical oxidation (photo-oxidation) in industrial large-scale production, the skilled person will take care the LED UV lighting or any array of LED UV lightings, or a multitude thereof, at least in total have sufficient power for industrial large-scale production, i.e., to initiate and maintain the photochemical oxidation (photo-oxidation).

The apparatus used for photochemical oxidations of the disclosure, thus, can be a Dow Corning photo reactor type G1 with an internal volume of 9 ml, as disclosed for example, on Dow Corning's website, and information contained therein for design and LEDs (www.corning.com/media/worldwide/Innovation/documents/G1photo_WEB.pdf). For example, Dow Corning's photo reactor type G1 is a construction of the irradiated glass channels (365 nm LEDs) as shown, for example, also in FIG. 8 of this disclosure.

The photo-oxidation according to the disclosure can be done in batch mode or continuous mode. As the photo-oxidation according to the disclosure is a gas phase reaction the continuous operating mode is preferred.

The photo-oxidation according to the disclosure comprises cooling of the reaction medium. Any cooling medium or heat transfer liquid, respectively, is suitable and can be used which let the wave length of the UV lighting source (e.g., of an Hg-lamp or LED) required for inducing and maintaining the photochemical reaction passing through the cooling medium. Water is the most preferred cooling medium and, for example, is applied as cooling medium if an Hg-lamp is used as the UV lighting source for the photo-oxidation. It is important to control the purity of the water applied for the cooling, e.g., in order to avoid reducing the performance of the photo-oxidation, which is caused by undesired photon absorption by impurities in the cooling medium. The same is valid for any other cooling/heat transfer liquids than water if used.

Regarding an Hg-lamp, the lamp consists out of two tubes, an outer tube and an inner tube, the lamp tube(s) being made out of quartz glass due to very high potential temperatures. The inner tube is containing the lamp with the vaporized Hg-cocoon, and the inner tube is floated with inert gas (mostly N₂) to avoid oxidation at the high voltage contacts of the lamp. The outer tube is a cooling tube for the lamp, and the cooling liquid for the lamp has to flow between inner lamp tube and outer cooling tube. The outer cooling tube is in contact with the reaction medium.

In respect of so-called light induced photo-oxidation there are two mechanistic types of photo-oxidation known in the related art, and the present disclosure can be implemented for each of the two mechanistic types of photo-oxidation:
a) Direct gas phase photo-oxidation with short UV wavelength which is most typically (mainly) the 254 nm line of an Hg-lamp or other available UV lighting source, equipment made out of quartz glass needs to be used to allow transmission of short UV wavelength to the reaction zone (starting material is activated directly), i.e., where the photo-oxidation takes place. There is a very high demand of photons.
b) A sensitized gas phase photo-oxidation using, e.g., elemental chlorine as light absorber (needed UV wavelength >290 nm); herein the generated chlorine radicals induce a radical chain photo-oxidation reaction which results in an energy saving as compared to before said “type a” direct gas phase photo-oxidation, and in most cases this “type b” sensitized gas phase photo-oxidation allows for higher yields and/or selectivity. This “type b” sensitized gas phase photo-oxidation results in a lower demand of photons (more energy saving).

For “type a” photo-oxidation (i.e., photo-oxidation without a sensitizer), the inner lamp tube is made out of quartz glass, and the outer cooling tube also has to be made out of quartz glass.

For “type b” photo-oxidation the inner lamp tube is made out of quartz glass, and the outer cooling tube in one aspect is made out of a borosilicate glass, for example, a Duran-50 glass or a Duran50/Pyrex/Borosilicate glass.

If in another aspect for type b photo-oxidations the outer cooling tube also is made out of quartz glass, an absorber solution liquid has to be used as cooling liquid to filter out all short UV wavelength(s) and to allow passing through only longer UV wavelength(s) of >290 nm. An overview about suitable lamp types, glass types and absorber solutions can be found in “Photochemie: Konzepte, Methoden, Experimente,” ISBN: 9783527660889. Avoiding said short UV wavelength(s) of an Hg-lamp in “type b” photo-oxidation significantly increases the yield and selectivity. “Type b” photo-oxidation, performed according to the related art process and using related art equipment (i.e., without such internal installations as proposed here by the present disclosure) of trichloroethylene to DCAC is disclosed in EP833810 and for fluorinated materials also in U.S. Pat. No. 5,259,938.

For “type a” photo-oxidation, a C—Cl bond of starting material, e.g., the C—Cl bond of HCFC-123, must be stimulated. This is done with a 254 nm line of a Hg-lamp (through quartz glass as filter) or 254 nm line of other UV lighting source. For a “type b” photo-oxidation the UV wavelengths are needed which are absorbed by the added sensitizer Cl₂ (chlorine gas), e.g., the greatest possible overlap between the emission spectrum of the UV lighting source and the absorption spectrum of the sensitizing Cl₂ (chlorine gas) is adjusted.

Whereas the interior parts of a photochemical reactor always has to be made out of a glass type material, the exterior parts of a photochemical reactor, for example, with the heat exchanger function and horizontal push, can be different material, and typically is made out of a corrosion resistant metal as very corrosive intermediates formed during photo-oxidation otherwise will cause strong corrosion. Nickel metal is the most preferred material of construction for exterior parts, and also all Hastelloys materials (such as known in the related art) also will fit for most of the photo-oxidations. For high grade stainless steel (e.g. 1.4571), which also may be used, however some corrosion will happen, especially in industrial and longer term usage scale, if (traces) of moisture might come into contact with the reactor surrounding, even if (traces) of moisture are removed towards the reactor exit by reaction with formed acetyl chlorides (besides the corrosion caused by the always formed HCl).

In one aspect of the disclosure, when performing the photo-oxidation, in a photochemical reactor, typically in an industrial photochemical reactor, with exterior parts made out of metal, typically out of nickel metal, interior installations inside the reactor force reaction media sequentially to pass from left to right and opposite (or the other way round to sequentially pass from right to left and opposite) over cooling/heat exchanger tubes/pipes (4) to get rid of the hot spots. This significantly increases yields and selectivity. Reference is made to FIG. 3, showing that internal installations (5) in the photochemical reactor forces reaction medium to pass over heat exchanger pipes (inner and outer tube details of Hg-lamp not drawn). An undesired short cut (e.g., distant vertical or distant parallel, respectively) passing-by of the reaction medium in the area at the outer lamp cooling tube and not passing-by the area at heat exchanger tubes/pipes is avoided, in this aspect of the present disclosure, by means of special plates (baffle plates) around the outer lamp cooling tube in between at each horizontally arranged plate, i.e., here the horizontal interior installations (5), which together form a horizontal channel system (6a) thorough which the reaction medium is forced to flow. These plates (baffle plates) typically can be made out of a metal resistant to the conditions of photochemical reaction, e.g., resistant to photochemical; typically the metal is nickel (Ni). Accordingly, the plate(s) (baffle plates) in a more preferred aspect of the disclosure is a nickel plate, i.e., a nickel baffle plate.

The plates (baffle plates) can also be made out of fluorinated polymers like FFKM (e.g., Kalrez®) or HDPTFE (high density polytetrafluoroethylene). Fluorinated polymers like those derived from PE (polyethylene) and PET (polyethylene terephthalate or poly(ethylene terephthalate) are less suitable, as PE and PET will swell in contact with the reaction medium and loose sealing properties after short time.

As shown in FIG. 3, for example, a horizontal channel system or channel path, respectively, is provided in the interior of the photochemical reactor, typically in the industrial photochemical reactor, by an assembly of horizontally arranged plates, i.e., an assembly of horizontal interior installations (5), by which horizontal channel system or channel path, respectively, the reaction medium is forced to sequentially to pass from left to right and opposite (or the other way round to sequentially pass from right to left and opposite) the area at the outer lamp cooling tube between two adjacent plates, i.e., here the horizontal interior installations (5), which together form a channel system (6a), and over vertically arranged cooling/heat exchanger tubes/pipes (4) to get rid of the hot spots.

In another aspect of the disclosure, when performing the photo-oxidation, in a photochemical reactor, typically in an industrial photochemical reactor, the one or more channels (6) inside the photochemical reactor reservoir can be construed by one or more interior installations (4) which are in the form of a coil, e.g. a glass or plastic coil, arranged around a UV lighting source (which can be an Hg-lamp or LED-lamp, or even a laser) of a photochemical reactor. For exemplification reference is made to FIG. 4, showing a simplified or schematic, respectively, coil reactor, meaning that the reaction medium is flowing through and the photo-oxidation is taking place in the channel (6) provided by the interior installation (4) in form of a coiled tube/pipe, which is placed in the reactor reservoir (2) comprising the cooling medium, and which is placed around the Hg-lamp (inner and outer tube details of Hg-lamp not drawn).

The coil reactor, i.e., the interior installation (4) in form of a coiled tube/pipe, wherein the photochemical oxidation reaction takes place, as exemplified in FIG. 4, is wrapped around the exterior cooling tube of the Hg-lamp. Normally, there will be a space of about 1 cm to about 2 cm between coil reactor and the Hg-lamp's outer cooling tube. Such a coil reactor is perfectly surrounded by cooling media and thus assuring an excellent heat exchange.

If for photo-oxidation “type a” the coil reactor is made out of quartz glass the coil's shape is rectangular, because quartz glass cannot be bent into a round form.

For photo-oxidation “type b” a coil reactor made out of borosilicate glass, e.g., out of Duran50 or Pyrex borosilicate glass, is typically used, or if desired even a plastic coil reactor which has to be transparent for UV wavelength(s) about >290 nm can be used. Transparent plastic materials suitable for such plastic coil reactor, for example, are fluorinated plastics in general and especially FEP. FEP is resistant to all the halogenated starting materials and also to the formed halogenated products. Such type of FEP tubes/pipes are commercially available, e.g., from Taizhou Chenguang Plastic Industry Co., Ltd.. The preferred diameter of such plastic coil reactor is about 0.5 cm to about 3 cm.

Depending on the power of the lamp used for the photo-oxidation of the present disclosure, also an assembly of several layers of coil reactor is possible, e.g. for allowing complete usage of all photons emitted by the lamp.

Regarding the sensitized photo-oxidation (“type b”), typically the largest possible overlap should be applied between the emission spectrum of the lamp used and the absorption spectrum of chlorine that is used as a sensitizer. More typically, it is noted that the largest possible overlap above a UV wavelength of about 290 nm is decisive; for example, with the condition that short UV wavelengths of the lamp are filtered out and only light of UV wavelength above 290 nm is allowed to pass through. Under such conditions, the product resulting from photo-oxidation then does not further react with oxygen. According to a preferred aspect of the disclosure, borosilicate glass is ideally suited as a filter, and thus preferred glass type.

Regarding an example of dimensions of photochemical reactors the following guidance is given, but if desired may be adapted by the person skilled in the art as deemed appropriate.

For example, a 200 l photochemical reactor with a 40 kW lamp is approx. 4 m high (including the lamp head) and is approx. 2 m wide.

The larger the photochemical reactor capacity, the narrower the baffle plates (e.g., the horizontal plates (5), as shown in FIGS. 3 and 5B) should be so that a very good heat exchange and very good mixing is ensured. As a result, for example, for a reactor with a 40 kW lamp, normally it is recommended by the present disclosure that the baffle plates (e.g., the horizontal plates (5), as shown in FIGS. 3 and 5B) for tube-/pipe-like formation of channels have a parallel distance to the adjacent ones of from about 1 cm up to about 10 cm.

As a result, for example, for a 200 l photochemical reactor with a 40 kW lamp and approx. 4 m height (including the lamp head) and approx. 2 m width, in an example, is about 5 cm.

As a result, for example, for a 400 ml laboratory photochemical reactor normally it is recommended by the present disclosure that the baffle plates (e.g., the horizontal plates (5), as shown in FIGS. 3 and 5B) for tube-/pipe-like formation of channels have a parallel distance to the adjacent ones of up to about 10 cm.

The dimension of channels (i.e., of the microtube(s)/micropipe(s)) in a photochemical microreactor is in the mm range. For example, the width of such channels (i.e., of the microtube(s)/micropipe(s)) in a photochemical microreactor are in the range of about 1 to 10 mm, typically in the range of about 1 to 8 mm, more typically in the range of about 1 to 5 mm. The width of such channels (i.e., of the microtube(s)/micropipe(s)) in a photochemical microreactor are in the range of about 1 to 3 mm or of about 1 to 2 mm.

For example, particular photochemical reactions, typically photochemical oxidations (photo-oxidations) performed according to certain aspects of the present disclosure are shown in the following reaction schemes:

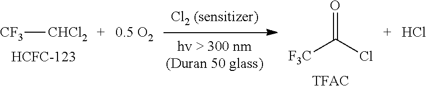

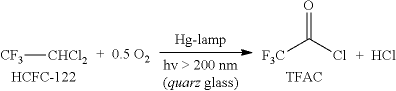

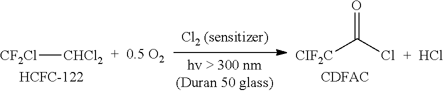

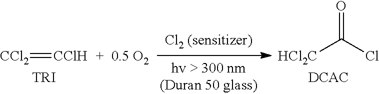

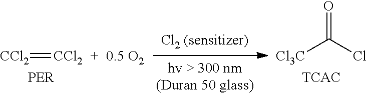

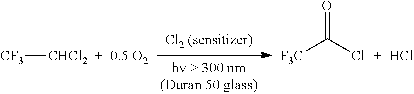

### HCFC-123 to TFAC/Coil Reactor, e.g., FEP-Coil Reactor.

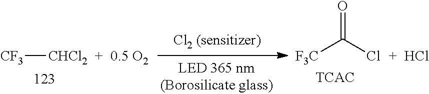

### Photochemical Microreactor.

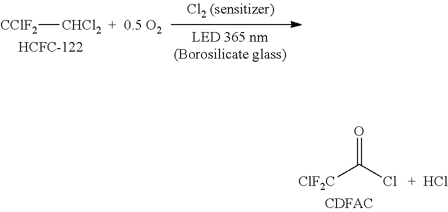

### Photochemical Microreactor.

In one aspect the disclosure relates to a photochemical reactor, comprising
at least one UV lighting source (1) providing UV lighting emission in the range of short-wavelength UV light to long-wavelength UV light of from about 100 nm to about 400 nm, typically of from about >200 to about 400 nm or of from about >290 to about 400 nm;
a reactor reservoir (2), surrounded by reactor wall (2a);
one or more inlet(s)/outlet(s) installation(s) (3a) and (3b);
and one or more interior installations (4) each forming a channel (6) inside the reactor reservoir (2), wherein the one or more interior installations (4) are in tube/pipe form and consist out of borosilicate glass and/or out of a quartz glass, and wherein each of the interior installations (4) at both tube/pipe ends have an inlet/outlet installation (4a) and (4b) extending to the exterior of the reactor reservoir (2);
   for example, the one or more interior installations (4) each forming a channel (6) inside the reactor reservoir (2), e.g., as shown in FIGS. 1 and 2, in one aspect of the disclosure are fixed within the reactor reservoir (2) by gasket(s);
and
optionally two or more interior installations (5) in plate form inside the reactor reservoir (2), wherein the two or more interior installations (5) are horizontally arranged in relation to the one or more interior installations (4) and in relation to the (upright) height of the reactor reservoir (2), and wherein the two or more interior installations (5) divide the reactor reservoir (2) into a further channel system (6a), each channel starting/ending at one of the one or more inlet(s)/outlet(s) installation(s) (3a) and (3b).

Herein, the one or more interior installations (4) each forming a channel (6) inside the reactor reservoir (2), e.g., as shown in FIGS. 3 and 5A, in one aspect of the disclosure are fixed within the reactor reservoir (2) either by gasket(s) or the gasket(s) are replaced by the two or more interior installations (5) in plate form inside the reactor reservoir (2). That is to say in other words, the two or more interior installations (5) in plate form inside the reactor reservoir (2) have also the function of gasket(s).

The number of the interior installations (5) depends on the size, e.g., of the height of the reactor reservoir (2), as further described herein below. The values given here under are typically for a 200 l reactor media volume, and give guidance for adaptations desired or found suitable by a person skilled in the art, for a smaller or a larger reactor media volume than the representative values given for a 200 l reactor media volume. For example, in one aspect there are two or more interior installations (5) in plate form inside the reactor reservoir (2) per twenty cm (20 cm) height of the reactor reservoir (2). For example, in another aspect the number of the two or more interior installations (5) in plate form inside the reactor reservoir (2) depends on the height of the reactor reservoir (2), and accordingly on the desired diameter (or height) of the further channel system (6a) formed between two adjacent interior installations (5) in plate form inside the reactor reservoir (2).

In other words, the distance between two adjacent interior installations (5) in plate form inside the reactor reservoir (2) depends on the height of the reactor reservoir (2), and the desired diameter (or height) of the further channel system (6a) formed between two adjacent interior installations (5) in plate form inside the reactor reservoir (2). For example, in one aspect, the distance between two adjacent interior installations (5) in plate form inside the reactor reservoir (2) of from about 1 to 10 cm, of from about 1 to 9 cm, of from about 1 to 8 cm, of from about 1 to 7 cm, of from about 1 to 6 cm, of from about 2 to 8 cm, of from about 2 to 7 cm, of from about 2 to 6 cm, of from about 3 to 8 cm, of from about 3 to 7 cm, of from about 3 to 6 cm, of from about 4 to 8 cm, of from about 4 to 7 cm, of from about 4 to 6 cm. In an example the distance between two adjacent interior installations (5) in plate form inside the reactor reservoir (2) is about 4 to 6 cm, e.g., said distance is about 5 cm.

In one aspect the disclosure relates to a photochemical reactor, typically according to the definition(s) given here before, comprising
at least one UV lighting source (1) providing UV lighting emission in the range of short-wavelength UV to long-wavelength UV of from about 100 nm to about 400 nm, typically of from about >200 to about 400 nm or of from about >290 to about 400 nm;
a reactor reservoir (2), surrounded by reactor wall (2a);
one or more inlet(s)/outlet(s) installation(s) (3a) and (3b);
and one or more interior installations (4) each forming a channel (6) inside the reactor reservoir (2), wherein the one or more interior installations (4) are in tube/pipe form and consist out of borosilicate glass and/or out of a quartz glass, and wherein each of the interior installations (4) at both tube/pipe ends have an inlet/outlet installation (4a) and (4b) extending to the exterior of the reactor reservoir (2).

For example, the one or more interior installations (4) each forming a channel (6) inside the reactor reservoir (2), e.g., as shown in FIGS. 1 and 2, in one aspect of the disclosure are fixed within the reactor reservoir (2) by gasket(s).

As regards to the shape of the one or more interior installations (4) in tube/pipe form, they may take any form as long as a channel (6) is provided thereby. For example, the disclosure also relates to a photochemical reactor according to the definition(s) given here before, wherein the one or more interior installations (4) in tube/pipe form are a linear (straight), bent, curved, snake shaped, sloped, zigzag or coiled tube/pipe, or combinations thereof. If quartz glass is used apparatus parts, e.g., such as tube(s) or pipe(s), normally will have an angular, angled, square, square-cut, square-edged or edgy or similarly shaped form, because quartz cannot be bent. Hence, for example, apparatus parts normally will be assembled out of quartz glass plane glass sheets of quartz.

As regards to the location of the one or more interior installations (4) in tube/pipe form, they may be located or distributed, respectively, within the reactor reservoir (2) as deemed appropriate in relation to the UV lighting source. For example, the disclosure also relates to a photochemical reactor according to the definition(s) given here before, wherein the one or more interior installations (4) arranged around the UV lighting source inside the reactor reservoir (2); for example, in one aspect of the disclosure arranged around a mercury-vapor lamp (Hg-lamp) (1).

As regards to the location of the one or more interior installations (4) in tube/pipe form, they may be orientated within the reactor reservoir (2) as deemed appropriate in relation to the UV lighting source. For example, the disclosure also relates to a photochemical reactor according to the definition(s) given here before, wherein the one or more interior installations (4) are arranged in vertical (parallel) or in horizontal direction in relation to the bottom surface of the reactor reservoir (2), and with an (upright) direction of the UV lighting source inside the reactor reservoir (2). For example, in one aspect of the disclosure the one or more interior installations (4) are arranged in vertical (parallel) or in horizontal direction in relation to the bottom surface of the reactor reservoir (2), and with an (upright) direction to a mercury-vapor lamp (Hg-lamp) (1) inside the reactor reservoir (2). For example, in an alternative aspect the disclosure also relates to a photochemical reactor according to the definition(s) given here before, wherein the one or more interior installations (4) are arranged inside the reactor reservoir (2) in vertical (parallel) direction in relation to the UV lighting source inside the reactor reservoir (2). For example, in one aspect of the disclosure the one or more interior installations (4) are arranged in vertical (parallel) direction in relation to the bottom surface of the reactor reservoir (2), and with an (upright) direction of a mercury-vapor lamp (Hg-lamp) (1) inside the reactor reservoir (2).

The disclosure also relates to a photochemical reactor according to the definition(s) given here before, wherein the one or more interior installations (4) are arranged in vertical (parallel) direction in relation to the bottom surface of the reactor reservoir (2), and with an (upright) direction of the UV lighting source inside the reactor reservoir (2), and the interior installations (4) are in a linear (straight) tube/pipe form. For example, in one aspect of the disclosure the one or more interior installations (4) are arranged in vertical (parallel) direction in relation to the bottom surface of the reactor reservoir (2), and with an (upright) direction of the UV lighting source inside the reactor reservoir (2), and the interior installations (4) are in a linear (straight) tube/pipe form. For example, in one aspect of the disclosure the one or more interior installations (4) are arranged in vertical (parallel) direction in relation to the bottom surface of the reactor reservoir (2), and with an (upright) direction of a mercury-vapor lamp (Hg-lamp) (1) inside the reactor reservoir (2), and the interior installations (4) are in a linear (straight) tube/pipe form.

In one aspect the disclosure also relates to the photochemical reactor as defined here before, wherein the one or more interior installations (4) inside the reactor reservoir (2), are connected at their outside with each other and/or held in place by one or more horizontal gasket(s). For example, in one aspect the disclosure relates to a photochemical reactor as defined here before, wherein the one or more interior installations (4) are arranged in a linear (straight) tube/pipe form and in vertical (parallel) direction in relation to the bottom surface of the reactor reservoir (2), and with an (upright) direction of a mercury-vapor lamp (Hg-lamp) (1) inside the reactor reservoir (2), and wherein the interior installations (4) are connected at their outside with each other and/or held in place by one or more horizontal gasket(s). For example, the one or more horizontal gasket(s) can be made out of fluorinated polymeric material. For example, the fluorinated polymeric material or “plastic material, as used here in the context of the disclosure, can be a fluorinated polymeric material which is transparent for a UV wavelength as applied in a photochemical reaction according to the disclosure, typically as applied in a photo-oxidation, and which fluorinated polymeric material is resistant to the reaction conditions. Particular examples of such the fluorinated polymeric material or plastic material, respectively, are the fluorinated polymeric materials as mentioned herein above, for example: PTFE, HDPTFE, FKM, FFKM, and/or FEP.

In another aspect the disclosure relates to a photochemical reactor, wherein the one or more interior installations (4) are arranged in a linear (straight) tube/pipe form and in vertical (parallel) direction in relation to the bottom surface of the reactor reservoir (2), and with an (upright) direction of the UV lighting source inside the reactor reservoir (2), and wherein the interior installations (4) are connected at their outside with each other and/or held by one or more horizontal plates (5). For example, in one aspect of the disclosure the one or more interior installations (4) are arranged in a linear (straight) tube/pipe form and in vertical (parallel) direction in relation to the bottom surface of the reactor reservoir (2), and with an (upright) direction of a mercury-vapor lamp (Hg-lamp) (1) inside the reactor reservoir (2), and wherein the interior installations (4) are connected at their outside with each other and/or held by one or more horizontal plates (5).

The disclosure, for example, here also relates to a photochemical reactor as defined here before, wherein typically the horizontal plates (5) are two or more interior installations (5) in plate form inside the reactor reservoir (2), wherein the two or more interior installations (5) are horizontally arranged in relation to the one or more interior installations (4) and in relation to the (upright) height of the reactor reservoir (2), and wherein the two or more interior installations (5) divide the reactor reservoir (2) into a further channel system (6a), each channel starting/ending at one of the one or more inlet(s)/outlet(s) installation(s) (3a) and (3b).

The disclosure, for example, here also relates to a photochemical reactor as defined here before, wherein the one or more horizontal plates (5) is a baffle plate(s) (5). For example, in one aspect here the disclosure also relates to a photochemical reactor as defined here before, wherein the one or more horizontal plates (5) is a baffle plate(s) (5), and typically the baffle plate(s) (5) are replacing the gasket(s) (not shown in the drawings), and in addition to forming the further channel system (6a) then are functioning also as gasket(s).

In still another aspect the disclosure relates to a photochemical reactor as defined here before, wherein the interior installation (4) is a single interior installation (4) in a coiled tube/pipe form and is arranged in vertical (parallel) direction in relation to the direction of the UV lighting source, and wherein its coiled tube/pipe form is surrounding the UV lighting source. For example, in one aspect of the disclosure the interior installation (4) is a single interior installation (4) in a coiled tube/pipe form and is arranged in vertical (parallel) direction in relation to the bottom surface of the reactor reservoir (2), and with an (upright) direction of a mercury-vapor lamp (Hg-lamp) (1), and wherein its coiled tube/pipe form is surrounding the mercury-vapor lamp (Hg-lamp) (1).

The disclosure also relates to a photochemical reactor as defined herein before, wherein the reactor reservoir (2) is a photochemical reaction zone and the interior installation (4) is a cooling medium zone. This arrangement is preferred if the photochemical reactor comprises one or more horizontal plates (5).

The disclosure also relates to a photochemical reactor as defined herein before, wherein the one or more interior installations (4) are arranged in a linear (straight) tube/pipe form and in vertical (parallel) direction in relation to the UV lighting source and are connected at their outside with each other and/or held in place by one or more horizontal plates (5). For example, in one aspect of the disclosure the one or more interior installations (4) are arranged in a linear (straight) tube/pipe form and in vertical (parallel) direction in relation to the bottom surface of the reactor reservoir (2), and with an (upright) direction of a mercury-vapor lamp (Hg-lamp) (1) and are connected at their outside with each other and/or held in place by one or more horizontal plates (5). In these arrangements it is preferred that in the photochemical reactor the reactor reservoir (2) is a photochemical reaction zone and the interior installation (4) is a cooling medium zone.

Accordingly, the disclosure also relates to a photochemical reactor as defined herein before, wherein the reactor reservoir (2) is a photochemical reaction zone and the interior installation (4) is a cooling medium zone, and wherein the one or more interior installations (4) are arranged in a linear (straight) tube/pipe form and in vertical (parallel) direction in relation to the UV lighting source and are connected at their outside with each other and/or held in place by one or more horizontal plates (5). For example, in one aspect of the disclosure the reactor reservoir (2) is a photochemical reaction zone and the interior installation (4) is a cooling medium zone, and the one or more interior installations (4) are arranged in a linear (straight) tube/pipe form and in vertical (parallel) direction in relation to the bottom surface of the reactor reservoir (2), and with an (upright) direction of a mercury-vapor lamp (Hg-lamp) (1) and are connected at their outside with each other and/or held in place by one or more horizontal plates (5).

The disclosure further relates to a photochemical reactor as defined herein before, wherein the interior installation (4) is a photochemical reaction zone and the reactor reservoir (2) is a cooling medium zone. This arrangement is preferred if the photochemical reactor comprises one or more interior installation (4) in a coiled tube/pipe form and is arranged in vertical (parallel) direction in relation to the direction of the UV lighting source, and wherein its coiled tube/pipe form is surrounding the UV lighting source, e.g., surrounding a mercury-vapor lamp (Hg-lamp) (1). Typically, this arrangement is realized with a coil reactor type.

The disclosure also relates to a photochemical reactor as defined herein before, wherein the interior installation (4) is a single interior installation (4) in a coiled tube/pipe form and is arranged in vertical (parallel) direction in relation to the direction of the UV lighting source, and wherein its coiled tube/pipe form is surrounding the UV lighting source. For example, in one aspect of the disclosure the interior installation (4) is a single interior installation (4) in a coiled tube/pipe form, and the single interior installation (4) in a coiled tube/pipe form is arranged in vertical (parallel) direction in relation to the bottom surface of the reactor reservoir (2), and with an (upright) direction of a mercury-vapor lamp (Hg-lamp) (1), and wherein its coiled tube/pipe form is surrounding the mercury-vapor lamp (Hg-lamp) (1). In these arrangements it is preferred that in the photochemical reactor the interior installation (4) in a coiled tube/pipe form is a photochemical reaction zone and the reactor reservoir (2) is a cooling medium zone.

Accordingly, the disclosure further relates to a photochemical reactor as defined herein before, wherein the interior installation (4) is a photochemical reaction zone and the reactor reservoir (2) is a cooling medium zone, and wherein the interior installation (4) is a single interior installation (4) in a coiled tube/pipe form and is arranged in vertical (parallel) direction in relation to the direction of the UV lighting source, and wherein its coiled tube/pipe form is surrounding the UV lighting source. For example, in one aspect of the disclosure the interior installation (4) is a photochemical reaction zone and the reactor reservoir (2) is a cooling medium zone, and the interior installation (4) is a single interior installation (4) in a coiled tube/pipe form and is arranged in vertical (parallel) direction in relation to the bottom surface of the reactor reservoir (2), and with an (upright) direction of a mercury-vapor lamp (Hg-lamp) (1), and wherein its coiled tube/pipe form is surrounding the mercury-vapor lamp (Hg-lamp) (1).

In a further aspect the disclosure relates to a photochemical reactor as defined herein before, wherein from the one or more inlet(s)/outlet(s) installation(s) (3a) and (3b) of the interior installation(s) (3):
(i) the installation(s) (3a) are inlet(s) (3a) and the installation(s) (3b) are outlet(s) (3b); or
(ii) the installation(s) (3a) are outlet(s) (3a) and the installation(s) (3b) are inlet(s) (3b).

In a further aspect the disclosure relates to a photochemical reactor as defined herein before, wherein there is only a single of each inlet(s)/outlet(s) installation(s) (3a) and (3b), and wherein:
(i) the installation (3a) is inlet (3a) and the installation (3b) is outlet (3b); or
(ii) the installation (3a) is outlet (3a) and the installation (3b) is inlet (3b).

In a further aspect the disclosure relates to a photochemical reactor as defined herein before, wherein from the one or more inlet(s)/outlet(s) installation(s) (4a) and (4b) of the interior installation(s) (4) are extending to the exterior of the reactor reservoir (2), and wherein:
(i) the installation(s) (4a) are inlet(s) (4a) and the installation(s) (4b) are outlet(s) (4b); or
(ii) the installation(s) (4a) are outlet(s) (4a) and the installation(s) (4b) are inlet(s) (4b).

In a further aspect the disclosure relates to a photochemical reactor as defined herein before, wherein the interior installation(s) (4) is a single interior installation (4) in a coiled tube/pipe form and is arranged in vertical (parallel) direction in relation to the bottom surface of the reactor reservoir (2), and with an (upright) direction of a mercury-vapor lamp (Hg-lamp) (1), and wherein its coiled tube/pipe form is surrounding the mercury-vapor lamp (Hg-lamp) (1), and there is only a single of each inlet(s)/outlet(s) installation(s) (4a) and (4b) extending to the exterior of the reactor reservoir (2), and wherein:
(i) the installation (4a) is inlet (4a) and the installation (4b) is outlet (4b); or
(ii) the installation (4a) is outlet (4a) and the installation (4b) is inlet (4b).

In another aspect the disclosure relates to a process for performing a photochemical oxidation of an organic compound, wherein the organic compound is reacted with oxygen in the gas phase by light induced photochemical oxidation by
a) direct gas phase photochemical oxidation (“type a” light induced photochemical oxidation) with short UV wavelength (2) of greater than 200 nm (but up to no more than 400 nm, typically up to no more than 290 nm) of a mercury-vapor lamp (Hg-lamp), typically with short UV wavelength (2) of mainly about 254 nm line of a mercury-vapor lamp (Hg-lamp), or by
b) sensitized gas phase photochemical oxidation (“type b” light induced photochemical oxidation) with the addition of elemental chlorine (sensitizer/light absorber), activating irradiation with light of UV wavelength (λ) of greater than 290 nm (but up to no more than 400 nm) of a mercury-vapor lamp (Hg-lamp);
   and wherein the light induced photochemical oxidation is performed in a photochemical reactor as defined herein before.

In regard to said process for performing a photochemical oxidation of an organic compound, wherein the organic compound is reacted with oxygen in the gas phase by light induced photochemical oxidation, as defined herein before, in a further aspect the disclosure relates to a photochemical reactor as defined herein before, wherein from the one or more inlet(s)/outlet(s) installation(s) (3a) and (3b) of the interior installation(s) (3):
(i) the installation(s) (3a) are inlet(s) (3a) and the installation(s) (3b) are outlet(s) (3b); or
(ii) the installation(s) (3a) are outlet(s) (3a) and the installation(s) (3b) are inlet(s) (3b).

In regard to said process for performing a photochemical oxidation of an organic compound, wherein the organic compound is reacted with oxygen in the gas phase by light induced photochemical oxidation, as defined herein before, in a further aspect the disclosure relates to a photochemical reactor as defined herein before, wherein there is only a single of each inlet(s)/outlet(s) installation(s) (3a) and (3b), and wherein:
(i) the single installation (3a) is a single inlet (3a) and the single installation (3b) is a single outlet (3b); or
(ii) the single installation (3a) is a single outlet (3a) and the single installation (3b) is a single inlet (3b).

In regard to said process for performing a photochemical oxidation of an organic compound, wherein the organic compound is reacted with oxygen in the gas phase by light induced photochemical oxidation, as defined herein before, in a further aspect the disclosure relates to a photochemical reactor as defined herein before, wherein from the one or more inlet(s)/outlet(s) installation(s) (4a) and (4b) of the interior installation(s) (4) are extending to the exterior of the reactor reservoir (2), and wherein:
(i) the installation(s) (4a) are inlet(s) (4a) and the installation(s) (4b) are outlet(s) (4b); or
(ii) the installation(s) (4a) are outlet(s) (4a) and the installation(s) (4b) are inlet(s) (4b).

In regard to said process for performing a photochemical oxidation of an organic compound, wherein the organic compound is reacted with oxygen in the gas phase by light induced photochemical oxidation, as defined herein before, in a further aspect the disclosure relates to a photochemical reactor as defined herein before, wherein the interior installation(s) (4) is a single interior installation (4) in a coiled tube/pipe form and is arranged in vertical (parallel) direction in relation to the bottom surface of the reactor reservoir (2), and with an (upright) direction of a mercury-vapor lamp (Hg-lamp) (1), and wherein its coiled tube/pipe form is surrounding the mercury-vapor lamp (Hg-lamp) (1), and there is only a single of each inlet(s)/outlet(s) installation(s) (4a) and (4b) extending to the exterior of the reactor reservoir (2), and wherein:
(i) the single installation (4a) is a single inlet (4a) and the single installation (4b) is a single outlet (4b); or
(ii) the single installation (4a) is a single outlet (4a) and the single installation (4b) is a single inlet (4b).

In a further particular aspect the present disclosure relates to a process for manufacturing a polyfluorochloro- and/or perfluorocarboxylic acid chloride compound of formula R^{a}CR^{b}XC(O)Cl, wherein R^{a} is chlorine, fluorine or perfluorinated alkyl having 1 to 10 carbon atoms, and R^{b} is hydrogen, chlorine or, fluorine, and X is chlorine or fluorine, by performing a photochemical oxidation of an organic compound selected from the group consisting of
(i) a halogenoalkane compound of formula R¹CFXCHCl₂, wherein R¹ is fluorine, chlorine or perfluorinated alkyl having 1 to 10 carbon atoms, and X is chlorine or fluorine, and
(ii) a halogenoalkylene compound of formula R²CX═CHₙClₘ, wherein R² is fluorine, chlorine or perfluorinated alkyl having 1 to 10 carbon atoms, and X is chlorine or fluorine, and n is an integer of 0 to 1, and m is an integer of 1 to 2, and the sum of n and m is 2;
wherein
   the compound as defined in (i) or (ii) is reacted with oxygen in the gas phase by light induced photochemical oxidation by
   a) direct gas phase photochemical oxidation (“type a” light induced photochemical oxidation) with short UV wavelength (λ) of greater than 200 nm (but up to no more than 400 nm, typically up to no more than 300 nm) of a mercury-vapor lamp (Hg-lamp), typically with short UV wavelength (λ) of mainly about 254 nm line of a mercury-vapor lamp (Hg-lamp), or by
   b) sensitized gas phase photochemical oxidation (“type b” light induced photochemical oxidation) with the addition of elemental chlorine (sensitizer/light absorber), activating irradiation with light of UV wavelength (λ) of greater than 290 nm (but up to no more than 400 nm) of a mercury-vapor lamp (Hg-lamp);
and wherein the light induced photochemical oxidation is performed in a photochemical reactor as defined herein before.

In regard to the above said process for manufacturing a polyfluorochloro- and/or perfluorocarboxylic acid chloride compound of formula R^{a}CR^{b}XC(O)Cl, wherein R^{a} is chlorine, fluorine or perfluorinated alkyl having 1 to 10 carbon atoms, and R^{b} is hydrogen, chlorine or, fluorine, and X is chlorine or fluorine, by performing a photochemical oxidation of an organic compound selected from the group consisting of (i) a halogenoalkane compound of formula R¹CFXCHCl₂, as defined herein above, and (ii) a halogenoalkylene compound of formula R²CX═CHₙClₘ, as defined herein above, in a further aspect the disclosure relates to a photochemical reactor as defined herein before, wherein from the one or more inlet(s)/outlet(s) installation(s) (3a) and (3b) of the interior installation(s) (3):
(i) the installation(s) (3a) are inlet(s) (3a) and the installation(s) (3b) are outlet(s) (3b); or
(ii) the installation(s) (3a) are outlet(s) (3a) and the installation(s) (3b) are inlet(s) (3b).

In regard to the above said process for manufacturing a polyfluorochloro- and/or perfluorocarboxylic acid chloride compound of formula R^{a}CR^{b}XC(O)Cl, wherein R^{a} is chlorine, fluorine or perfluorinated alkyl having 1 to 10 carbon atoms, and R^{b} is hydrogen, chlorine or, fluorine, and X is chlorine or fluorine, by performing a photochemical oxidation of an organic compound selected from the group consisting of (i) a halogeno-alkane compound of formula R¹CFXCHCl₂, as defined herein above, and (ii) a halogeno-alkylene compound of formula R²CX═CHₙClₘ, as defined herein above, in a further aspect the disclosure relates to a photochemical reactor as defined herein before, wherein there is only a single of each inlet(s)/outlet(s) installation(s) (3a) and (3b), and wherein:
(i) the single installation (3a) is a single inlet (3a) and the single installation (3b) is a single outlet (3b); or
(ii) the single installation (3a) is a single outlet (3a) and the single installation (3b) is a single inlet (3b).

In regard to the above said process for manufacturing a polyfluorochloro- and/or perfluorocarboxylic acid chloride compound of formula R^{a}CR^{b}XC(O)Cl, wherein R^{a} is chlorine, fluorine or perfluorinated alkyl having 1 to 10 carbon atoms, and R^{b} is hydrogen, chlorine or, fluorine, and X is chlorine or fluorine, by performing a photochemical oxidation of an organic compound selected from the group consisting of (i) a halogenoalkane compound of formula R¹CFXCHCl₂, as defined herein above, and (ii) a halogeno-alkylene compound of formula R²CX═CHₙClₘ, as defined herein above, in a further aspect the disclosure relates to a photochemical reactor as defined herein before, wherein from the one or more inlet(s)/outlet(s) installation(s) (4a) and (4b) of the interior installation(s) (4) are extending to the exterior of the reactor reservoir (2), and wherein:
(i) the installation(s) (4a) are inlet(s) (4a) and the installation(s) (4b) are outlet(s) (4b); or
(ii) the installation(s) (4a) are outlet(s) (4a) and the installation(s) (4b) are inlet(s) (4b).

In regard to the above said process for manufacturing a polyfluorochloro- and/or perfluorocarboxylic acid chloride compound of formula R^{a}CR^{b}XC(O)Cl, wherein R^{a} is chlorine, fluorine or perfluorinated alkyl having 1 to 10 carbon atoms, and R^{b} is hydrogen, chlorine or, fluorine, and X is chlorine or fluorine, by performing a photochemical oxidation of an organic compound selected from the group consisting of (i) a halogeno-alkane compound of formula R¹CFXCHCl₂, as defined herein above, and (ii) a halogeno-alkylene compound of formula R²CX═CHₙClₘ, as defined herein above, in a further aspect the disclosure relates to a photochemical reactor as defined herein before, wherein the interior installation(s) (4) is a single interior installation (4) in a coiled tube/pipe form and is arranged in vertical (parallel) direction in relation to the bottom surface of the reactor reservoir (2), and with an (upright) direction of a mercury-vapor lamp (Hg-lamp) (1), and wherein its coiled tube/pipe form is surrounding the mercury-vapor lamp (Hg-lamp) (1), and there is only a single of each inlet(s)/outlet(s) installation(s) (4a) and (4b) extending to the exterior of the reactor reservoir (2), and wherein:
(i) the single installation (4a) is a single inlet (4a) and the single installation (4b) is a single outlet (4b); or
(ii) the single installation (4a) is a single outlet (4a) and the single installation (4b) is a single inlet (4b).

The disclosure in one aspect also relates to such a process for manufacturing a polyfluorochloro- and/or perfluorocarboxylic acid chloride compound as defined herein above, wherein in the compound of formula R^{a}CR^{b}XC(O)Cl manufactured by performing the photochemical oxidation the substituent R^{a} is chlorine or fluorine, and R^{b} is hydrogen, chlorine or, fluorine, and X is chlorine or fluorine.

The disclosure in one aspect also relates to such a process for manufacturing a polyfluorochloro- and/or perfluorocarboxylic acid chloride compound as defined herein above, wherein the compound of formula R^{a}CR^{b}XC(O)Cl manufactured by performing the photochemical oxidation is selected from the group consisting of trifluoroacetyl chloride (TFAC; the substituents R^{a}, R^{b} and X each are fluorine, i.e., the R^{a}CR^{b}XC-group represents a CF₃-group), chlorodifluoroacetyl chloride (CDFAC; the substituents R^{a} and R^{b} each are fluorine and X is chlorine, i.e., the R^{a}CR^{b}XC-group represents a CF₂Cl-group), trichloroacetyl chloride (TCAC; the substituents R^{a}, R^{b} and X each are chlorine, i.e., the R^{a}CR^{b}XC-group represents a CCl₃-group), and dichloroacetyl chloride (DCAC; the substituents R^{a} and X each are chlorine, and the substituent R^{b} is hydrogen, i.e., the R^{a}CR^{b}XC-group represents a CHCl₂-group).

The disclosure in one aspect also relates to such a process for manufacturing a polyfluorochloro- and/or perfluorocarboxylic acid chloride compound of formula R^{a}CR^{b}XC(O)Cl, as defined herein above, wherein the organic compound subjected to performing the photochemical oxidation is selected from the group consisting of
(i) the halogenoalkane compound of formula R¹CFXCHCl₂, wherein the substituent R¹ is fluorine or chlorine, and X is chlorine or fluorine, and
(ii) the halogenoalkylene compound of formula R²CX═CHₙClₘ, wherein the substituent R² is fluorine or chlorine, and X is chlorine or fluorine, and n is an integer of 0 to 1, and m is an integer of 1 to 2, and the sum of n and m is 2.

The disclosure in one aspect also relates to such a process for manufacturing a polyfluorochloro- and/or perfluorocarboxylic acid chloride compound of formula R^{a}CR^{b}XC(O)Cl, as defined herein above, wherein
(i) the halogenoalkane compound of formula R¹CFXCHCl₂ subjected to performing the photochemical oxidation is selected from the group consisting of 1,1,1-trifluoro-2,2-dichloroethane (HCFC-123) and 1,1-difluoro-1,2,2-trichloroethane (HCFC-122); or
(ii) the halogeno-alkylene compound of formula R²CX═CHₙClₘ subjected to performing the photochemical oxidation is selected from the group consisting of 1,1-dichloro-2-chloroethylene (TRI; the substituent R² and X each are chlorine, and n, m each are integer 1) and perchloroethylene (PER; the substituent R² and X each are chlorine, and n is 0, m is integer 2).

The disclosure in one aspect also relates to such a process for manufacturing a polyfluorochloro- and/or perfluorocarboxylic acid chloride compound of formula R^{a}CR^{b}XC(O)Cl, as defined herein above, wherein
(i) the halogeno-alkane compound of formula R¹CFXCHCl₂ subjected to performing the photochemical oxidation is
   (i-1) 1,1,1-trifluoro-2,2-dichloroethane (HCFC-123) and the compound manufactured by the photochemical oxidation thereof having formula R^{a}CR^{b}XC(O)Cl is trifluoroacetyl chloride (TFAC; the substituents R^{a}, R^{b} and X each are fluorine, i.e., the R^{a}CR^{b}XC-group represents a CF₃-group); or
   (i-2) 1,1-difluoro-1,2,2-trichloroethane (HCFC-122) and the compound manufactured by the photochemical oxidation thereof having formula R^{a}CR^{b}XC(O)Cl is chlorodifluoroacetyl chloride (CDFAC; the substituents R^{a} and R^{b} each are fluorine and X is chlorine, i.e., the R^{a}CR^{b}XC-group represents a CF₂Cl-group);
or
(ii) the halogenoalkylene compound of formula R²CX═CHₙClₘ subjected to performing the photochemical oxidation is
   (ii-1) 1,1-dichloro-2-chloroethylene (TRI) and the compound manufactured by the photochemical oxidation thereof having formula R^{a}CR^{b}XC(O)Cl is dichloroacetyl chloride (DCAC; the substituents R^{a} and X each are chlorine, and the substituent R^{b} is hydrogen, i.e., the R^{a}CR^{b}XC-group represents a CHCl₂-group); or
   (ii-1) perchloroethylene (PER) and the compound manufactured by the photochemical oxidation thereof having formula R^{a}CR^{b}XC(O)Cl is trichloroacetyl chloride (TCAC; the substituents R^{a}, R^{b} and X each are chlorine, i.e., the R^{a}CR^{b}XC-group represents a CCl₃-group).

In a further particular aspect the disclosure also relates to a process for performing a photochemical oxidation of an organic compound, wherein the organic compound is reacted with oxygen in the gas phase by light induced photochemical oxidation, wherein the light induced photochemical oxidation is performed in a photochemical microreactor (M) comprising
at least one array (1^{M}) of UV lighting sources (1^{M}a) providing UV lighting emission in the range of short-wavelength UV light to long-wavelength UV light of from about 100 nm to about 400 nm, typically of from about >200 nm up to about 400 nm, or from about >290 nm up to about 400 nm, or of from about >300 nm up to about 400 nm, and wherein the UV wavelength (λ) is adjusted to the UV wavelength (λ) for direct gas phase photochemical oxidation as defined above under a) or to the UV wavelength (λ) for sensitized gas phase photochemical oxidation as defined above under b), or is or adjustable thereto;
at least one reactor reservoir (2^{M}), surrounded by reactor wall (2^{M}a);
one or more inlet(s)/outlet(s) installation(s) (3^{M}a) and (3^{M}b);
and one or more interior installations (4^{M}) each forming a channel (6^{M}) inside the reactor reservoir (2^{M}), wherein the one or more interior installations (4^{M}) are in tube/pipe form and consist out of [borosilicate] glass, and wherein each of the interior installations (4^{M}) at both tube/pipe ends have an inlet/outlet installation (4^{M}a) and (4^{M}b) extending to the exterior of the reactor reservoir (2^{M}).

In still a further particular aspect the disclosure also relates to a process for performing a photochemical oxidation of an organic compound, wherein the organic compound is reacted with oxygen in the gas phase by light induced photochemical oxidation, wherein the light induced photochemical oxidation is performed in a photochemical microreactor (M), and wherein the organic compound is reacted with oxygen in the gas phase by light induced photochemical oxidation by
a) direct gas phase photochemical oxidation (“type a” light induced photochemical oxidation) with UV wavelength (λ) of greater than 200 nm (but up to no more than 400 nm), typically with UV wavelength (λ) of greater than 290 nm (but up to no more than 400 nm), or of greater than 300 nm (but up to no more than 400 nm), provided by at least one UV lighting source, more typically with UV wavelength (λ) of mainly about 365 nm line of LED UV lighting source (e.g., type G1, Dow Corning), or by
b) sensitized gas phase photochemical oxidation (“type b” light induced photochemical oxidation) with the addition of elemental chlorine (sensitizer/light absorber), activating irradiation with light of UV wavelength (λ) of greater than 290 nm (but up to no more than 400 nm), or of greater than 300 nm (but up to no more than 400 nm), typically with UV wavelength (λ) of greater than 300 nm (but up to no more than 400 nm), provided by at least one UV lighting source, more typically with UV wavelength (λ) of mainly about 365 nm line of LED UV lighting source (e.g., type G1, Dow Corning);
and wherein the light induced photochemical oxidation is performed in a photochemical microreactor (M) comprising
   at least one array (1^{M}) of UV lighting sources (1^{M}a) providing UV lighting emission in the range of short-wavelength UV light to long-wavelength UV light (of from about 100 nm to about 400 nm, typically of from about >200 to about 400 nm, or of from about >290 nm to about 400 nm, or of from about >300 nm to about 400 nm), and wherein the UV wavelength (λ) is adjusted to the UV wavelength (λ) for direct gas phase photochemical oxidation as defined above under a) or to the UV wavelength (λ) for sensitized gas phase photochemical oxidation as defined above under b), or is or adjustable thereto;
   at least one reactor reservoir (2^{M}), surrounded by reactor wall (2^{M}a);
   one or more inlet(s)/outlet(s) installation(s) (3^{M}a) and (3^{M}b);
   and one or more interior installations (4^{M}) each forming a channel (6^{M}) inside the reactor reservoir (2^{M}), wherein the one or more interior installations (4^{M}) are in tube/pipe form and consist out of [borosilicate] glass, and wherein each of the interior installations (4^{M}) at both tube/pipe ends have an inlet/outlet installation (4^{M}a) and (4^{M}b) extending to the exterior of the reactor reservoir (2^{M}).

In another aspect the disclosure also relates to such a process for performing a photochemical oxidation of an organic compound, wherein the organic compound is reacted with oxygen in the gas phase by light induced photochemical oxidation, wherein the light induced photochemical oxidation is performed in a photochemical microreactor (M), as defined herein above, wherein the one or more interior installations (4^{M}) each forming a channel (6^{M}) and the at least one array (1^{M}) of UV lighting sources (1^{M}a) together for an irradiated glass channel system.

In still a further aspect the disclosure also pertains to a novel process for the elimination of oxidizing by-products which possibly may be formed during a photochemical oxidation and possibly accumulate at certain parts and/or certain positions of a photochemical reactor system and/or process equipment used in context of a photochemical oxidation, for example, such equipment as cooling traps and piping systems.

Accordingly, in this further aspect the disclosure pertains to a process for performing a photochemical oxidation of an organic compound, wherein the organic compound is reacted with oxygen in the gas phase by light induced photochemical oxidation, as defined herein above, typically a process according and wherein any oxidizing material(s) and/or oxidizing by-product(s) formed in the photochemical oxidation, typically wherein the oxidizing material(s) and/or oxidizing by-product(s) is an organic peroxide compound derived from the organic compound that is reacted with oxygen in the gas phase by light induced photochemical oxidation, is eliminated from the reaction mixture obtained from the photochemical oxidation by contacting and/or treating said oxidizing material and/or oxidizing by-products with initiating material(s).

For example, in the aspect of the elimination of oxidizing by-products, the disclosure is directed to a process for performing a photochemical oxidation of an organic compound, wherein the organic compound is reacted with oxygen in the gas phase by light induced photochemical oxidation, wherein the initiating material(s)
(i) is a radical or free radical generating agent, and/or
(ii) is selected from the group consisting of carbon black steel or carbon steel, corroded metals, transition metal oxides wherein the metal is selected from the group consisting of iron, cobalt, copper, nickel, tungsten and zinc, transition metal chlorides wherein the metal is selected from the group consisting of iron, cobalt, copper, nickel, tungsten and zinc, and also including mixtures of said oxides, chlorides and/or mixtures of said oxides and chlorides, and of carbon, activated carbon, deactivated active carbon, and pre-treated active carbon.

In the aspect of the elimination of oxidizing by-products, typically the disclosure is directed to a process for performing a photochemical oxidation of an organic compound, wherein the organic compound is reacted with oxygen in the gas phase by light induced photochemical oxidation, wherein the initiating material(s) is selected from the group consisting of carbon black steel or carbon steel, corroded steel, iron oxide(s), cobalt oxide(s), copper oxide(s), nickel oxide(s), tungsten oxide(s), zinc oxide(s), iron chloride(s), cobalt chloride(s), copper chloride(s), nickel chloride(s), tungsten chloride(s), zinc chloride(s), and also including mixtures of said oxides, chlorides and/or mixtures of said oxides and chlorides, carbon, activated carbon, deactivated active carbon, and pre-treated active carbon.

Furthermore, in the aspect of the elimination of oxidizing by-products, typically the disclosure is directed to a process for performing a photochemical oxidation of an organic compound, wherein the organic compound is reacted with oxygen in the gas phase by light induced photochemical oxidation, wherein any oxidizing material(s) and/or oxidizing by-product(s),
typically wherein the oxidizing material(s) and/or oxidizing by-product(s) is an organic peroxide compound derived from the organic compound that is reacted with oxygen in the gas phase by light induced photochemical oxidation,
is eliminated from a reaction mixture obtained from a photochemical oxidation of the process as defined above for manufacturing a polyfluorochloro- and/or perfluorocarboxylic acid chloride compound of formula R^{a}CR^{b}XC(O)Cl as defined above.

In particular, in the aspect of the elimination of oxidizing by-products, typically the disclosure is directed to a process for performing a photochemical oxidation of an organic compound, wherein the organic compound is reacted with oxygen in the gas phase by light induced photochemical oxidation, as defined herein before, wherein the organic compound selected from the group consisting of
(i) a halogenoalkane compound of formula R¹CFXCHCl₂, wherein R¹ is fluorine, chlorine or perfluorinated alkyl having 1 to 10 carbon atoms, and X is chlorine or fluorine, and
(ii) a halogeno-alkylene compound of formula R²CX═CHₙClₘ, wherein R² is fluorine, chlorine or perfluorinated alkyl having 1 to 10 carbon atoms, and X is chlorine or fluorine, and n is an integer of 0 to 1, and m is an integer of 1 to 2, and the sum of n and m is 2.

In this aspect of the elimination of oxidizing by-products, typically the disclosure is directed to a process for performing a photochemical oxidation of an organic compound, wherein the organic compound is reacted with oxygen in the gas phase by light induced photochemical oxidation, as defined here before, wherein in the compound of formula R^{a}CR^{b}XC(O)Cl manufactured by performing the photochemical oxidation the substituent R^{a} is chlorine or fluorine, and R^{b} is hydrogen, chlorine or, fluorine, and X is chlorine or fluorine; typically wherein the compound of formula R^{a}CR^{b}XC(O)Cl manufactured by performing the photochemical oxidation is selected from the group consisting of trifluoroacetyl chloride (TFAC; the substituents R^{a}, R^{b} and X each are fluorine, i.e., the R^{a}CR^{b}XC-group represents a CF₃-group), chlorodifluoroacetyl chloride (CDFAC; the substituents R^{a} and R^{b} each are fluorine and X is chlorine, i.e., the R^{a}CR^{b}XC-group represents a CF₂Cl-group), trichloroacetyl chloride (TCAC; the substituents R^{a}, R^{b} and X each are chlorine, i.e., the R^{a}CR^{b}XC-group represents a CCl₃-group), and dichloroacetyl chloride (DCAC; the substituents R^{a} and X each are chlorine, and the substituent R^{b} is hydrogen, i.e., the R^{a}CR^{b}XC-group represents a CHCl₂-group).

More typically, in this aspect of the elimination of oxidizing by-products, the disclosure is directed to a process for performing a photochemical oxidation of an organic compound, wherein the organic compound is reacted with oxygen in the gas phase by light induced photochemical oxidation, as defined here before, wherein the organic compound is (i) a halogenoalkane compound of formula R¹CFXCHCl₂ as defined above and/or (ii) a halogeno-alkylene compound of formula R²CX═CHₙClₘ as defined above.

The disclosure also comprises a process for performing a photochemical oxidation of an organic compound wherein the organic compound is reacted with oxygen in the gas phase by light induced photochemical oxidation, as defined and described herein before, wherein the process further comprises passing the reaction mixture obtained by the photochemical reaction over a decomposition reactor, typically a decomposition reactor filled with carbon.

The following examples are intended to further illustrate the disclosure without limiting its scope.

### EXAMPLES

### Example 1 (Comparative)

“Type b” TFAC preparation out of HCFC-123 using Cl₂ as sensitizer; photo-oxidation in 400 ml laboratory apparatus, i.e., standard apparatus (Duran 50 glass, a borosilicate glass), with no “interior installations.”

According to Example 6 in EP 659729, a TQ 718 Hg-medium pressure lamp with 700 W power was used in a 400 ml photo reactor with borosilicate glass cooling tube and just compressed air as cooling media for the lamp (no interior installations, i.e., none such as proposed by the present disclosure). The HCFC-123 was vaporized in a vaporizer, heated to 100° C. and mixed with 0.6 eq. O₂ and 10 mol-% Cl₂ before the mixture entered as gas the photo reactor from the bottom. Samples were taken out of the gas stream leaving the reactor at the top. The HCFC-123 conversion was 75%, the selectivity to TFAC taken out of a sample from the gas stream with a so called gas mouse (20 ml sampling device) leaving the reactor was 98%, all values given in GC area % on a CP-SIL 8 column.

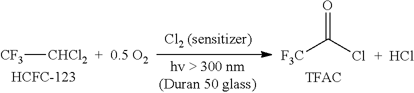

### Example 2 (Comparative)

“Type b” TFAC preparation out of HCFC-123 using Cl₂ as sensitizer; photo-oxidation in 200 l apparatus, i.e., standard apparatus (borosilicate glass), with no “interior installations.” See also reaction scheme shown in Example 1 (Comparative).

A 200 l reactor media volume (3 m length-without the electrical head for the lamp), 40 kW Hg-medium pressure lamp from Heraeus Noblelight, cooling tube made out of borosilicate glass, no interior installations (i.e., none such as proposed by the present disclosure). “Type b” TFAC preparation out of HCFC-123 using Cl₂ as sensitizer.

30 mol HCFC-123/h were vaporized at 60° C. and mixed with 0.52 eq. O₂ and 10 mol-% Cl₂ and fed into a photo reactor completely made out of glass. The conversion of HCFC-123 was 47%, the selectivity to TFAC 65%. 9% CFC-113a (1,1,1-trichloro-2,2,2-trifluoroethane) was detected as a result from chlorination instead of photo-oxidation. Besides that, 14% COCl₂, 5% COF₂ and 3% COFCl was found; all values given in GC area % on a CP-SIL 8 column.

### Example 3 (Disclosure)

“Type b” TFAC preparation out of HCFC-123 using Cl₂ as sensitizer; photo-oxidation in 200 l apparatus, i.e., apparatus (borosilicate glass) with “interior installations.”

See FIG. 3 and FIG. 5A for apparatus and reaction. See also reaction scheme shown in Example 1 (Comparative) which is applicable here in Example 3 (Disclosure), too.

A 200 l reactor media volume (3 m length-without the electrical head for the lamp), exterior made out of Ni, 40 kW Hg-medium pressure lamp from Heraeus Noblelight, cooling tube made out of borosilicate glass, interior installations (i.e., such as proposed by the present disclosure). “Type b” TFAC preparation out of HCFC-123 using Cl₂ as sensitizer.

A photo reactor with cooling tube made out of borosilicate glass, the lamp tube was made out of quartz glass, cooling water 25° C., interior Ni-plate every 5 cm as drawn in FIG. 3 and FIG. 5A. 30 mol HCFC-123/h were vaporized at 60° C. and mixed with 0.52 eq. O₂ and 10 mol-% (0.1 eq.) Cl₂ and fed into this photo reactor with exterior made out of nickel metal. The conversion of HCFC-123 was 89%, the selectivity to TFAC 99.5%. 0.2% CFC-113a (1,1,1-trichloro-2,2,2-trifluoroethane) and no phosgenes were detected; all values given in GC area % on a CP-SIL 8 column.

### Example 4 (Comparative)

“Type b” TFAC preparation out of HCFC-without sensitizer; photo-oxidation in 200 l cooling tube apparatus, i.e., standard apparatus (quartz glass), with no “interior installations.”

A 200 l reactor media volume (3 m length-without the electrical head for the lamp), 40 kW Hg-medium pressure lamp from Heraeus Noblelight, cooling tube made out of quartz glass, no interior installations (i.e., none such as proposed by the present disclosure), “Type a” TFAC preparation out of HCFC-123 without any sensitizer (according to Halocarbon, U.S. Pat. No. 3,883,407).

30 mol HCFC-123/h were vaporized at 60° C. and mixed with 0.52 eq. O₂ and 10 mol-% Cl₂ and fed into a photo reactor completely made out of glass. The conversion of HCFC-123 was only 32%; the selectivity to yield TFAC was 71%. 15% COCl₂ and 2% COF₂, and also 1% CO₂ was detected as a result from over-oxidation induced by insufficient mixing and hot spots; all values given in GC area % on a CP-SIL 8 column.

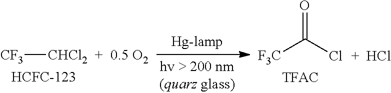

### Example 5 (Disclosure)

“Type a” TFAC preparation out of HCFC-123 without any sensitizer.

See FIG. 3 and FIG. 5A for apparatus and reaction. See also reaction scheme shown in Example 4 (Comparative) which is applicable here in Example 5 (Disclosure), too.

A 200 l reactor media volume (3 m length-without the electrical head for the lamp), exterior made out of Ni, 40 kW Hg-medium pressure lamp from Heraeus Noblelight, cooling tube also made out of quartz glass, interior installations (i.e., such as proposed by the present disclosure) in Ni-exterior reactor. “Type a” TFAC preparation out of HCFC-123 without any sensitizer.

A photo reactor with cooling tube made out of quartz glass, the lamp tube was made out of quartz glass, cooling water set to 25° C., interior Ni-plate every 5 cm as drawn in FIG. 3 and FIG. 5A. 30 mol HCFC-123/h were vaporized at 60° C. and mixed with 0.52 eq. O₂ and 10 mol-% (0.1 eq.) Cl₂ and fed into this photo reactor with exterior made out of nickel metal. The conversion of HCFC-123 was 49%, the selectivity to TFAC 93%. COCl₂ in the gas stream leaving the reactor was 5%, COFCl was present with 2% (no CO₂ was detectable!); all values given in GC area % on a CP-SIL 8 column.

### Example 6 (Disclosure)

“Type b” CDFAC preparation out of HCFC-122 using Cl₂ as sensitizer.

See FIG. 3 and FIG. 5A for apparatus and reaction.

A 200 l reactor media volume (3 m length-without the electrical head for the lamp), exterior made out of Ni, 40 kW Hg-medium pressure lamp from Heraeus Noblelight, cooling tube made out of Borosilicate glass, interior installations (i.e., such as proposed by the present disclosure). “Type b” CDFAC preparation out of HCFC-122 using Cl₂ as sensitizer.

A photo reactor with cooling tube made out of borosilicate glass, here a Duran 50 glass, the lamp tube was made out of quartz glass, cooling water 50° C., interior Ni-plate every 5 cm as drawn in FIG. 3 and FIG. 5A. 30 mol HCFC-122/h were vaporized at 120° C. and mixed with 0.52 eq. O₂ and 10 mol-% (0.1 eq.) Cl₂ and fed into this photo reactor with exterior completely made out of nickel metal. The gas stream leaving the reactor had a temperature of 68° C.

The conversion of HCFC-122 was 83%, the selectivity to CDFAC 96%. 0.1% CFC-112a (tetrachloro-1,1-difluoroethane), 2% COCl₂ and traces of COFCl were detected (no CO₂ was detectable!); all values given in GC area % on a CP-SIL 8 column.

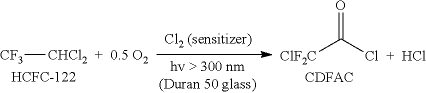

### Example 7 (Disclosure)

“Type b” DCAC preparation out of trichloroethylene (TRI) using Cl₂ as sensitizer.

See FIG. 3 and FIG. 5A for apparatus and reaction.

A 200 l reactor media volume (3 m length-without the electrical head for the lamp), exterior made out of Ni, 40 kW Hg-medium pressure lamp from Heraeus Noblelight, cooling tube made out of borosilicate glass, interior installations (i.e., such as proposed by the present disclosure). “Type b” DCAC preparation out of Trichloroethylene (TRI) using Cl₂ as sensitizer.

A photo reactor with cooling tube made out of borosilicate glass, here a Duran 50 glass, the lamp tube was made out of quartz glass, lamp cooling water 50° C., interior Ni-plate every 5 cm with side changing material transition holes as drawn in FIG. 3 and FIG. 5A. 30 mol TRI/h were vaporized at 150° C. and mixed with 0.52 eq. O₂ and 10 mol-% (0.1 eq.) Cl₂ and fed into this photo reactor with exterior completely made out of nickel metal. The gas stream leaving the reactor had a temperature of 110° C. The conversion of TRI was 87%, the selectivity to DCAC 96%. 2% pentachloroethane and 1% hexachloroethane was found besides 2% trichloroethylene epoxide-all values given in GC area % on a CP-SIL 8 column.

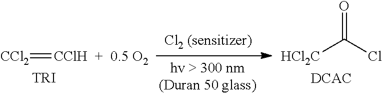

### Example 8 (Comparative)

“Type b” Trichloroacetylchloride (TCAC) preparation out of perchloroethylene (PER) with Cl₂-sensitation.

200 l reactor media volume (3 m length-without the electrical head for the lamp), 40 KW Hg-medium pressure lamp from Heraeus Noblelight, cooling tube made out of borosilicate glass, no interior installations (i.e., none such as proposed by the present disclosure). “Type b” trichloroacetylchloride (TCAC) preparation out of perchloroethylene (PER) with Cl₂-sensitation.

30 mol PER were vaporized at 150° C. and mixed with 0.8 eq. 02 and 10 mol-% C12 and fed (from top to bottom) into a photo reactor completely made out of glass. The conversion of PER was 45%, the selectivity to TCAC was 80%. 17% COCl₂ also 2% CO₂ was detected as a result from over-oxidation probably induced by insufficient mixing and hot spots-all values given in GC area % on a CP-SIL 8 column.

### Example 9 (Disclosure)

“Type b” TCAC preparation out of Perchloroethylene (PCE) using Cl₂ as sensitizer.

See FIG. 4 and FIG. 5B for apparatus and reaction.

A 200 l reactor media volume (3 m length-without the electrical head for the lamp), exterior made out of Ni, 40 kW Hg-medium pressure lamp from Heraeus Noblelight, cooling tube made out of borosilicate glass, interior installations (i.e., such as proposed by the present disclosure). “Type b” TCAC preparation out of perchloroethylene (PCE) using Cl₂ as sensitizer.

30 mol PER were vaporized at 150° C. and mixed with 0.6 eq. O₂ and 10 mol-% C12 and fed (from top to bottom) into a photo reactor with exterior made out of nickel. The conversion of PER was 92%, the selectivity to TCAC was 98%; 2% COCl₂ also were formed; all values given in GC area % on a CP-SIL 8 column.

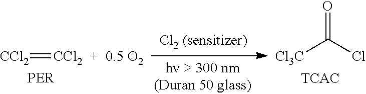

### Example 10 (Disclosure)

“Type b” TFAC preparation out of HCFC-123 using Cl₂ as sensitizer and FEP-coil reactor, wherein the FEP-coil is an interior installation according to the disclosure.

See FIG. 4 and FIG. 5B for apparatus and reaction.

A 400 ml lab apparatus, a FEP coil (supplier: OPTUBUS GmbH, www.optubus.de) with internal diameter of 1 cm and 1.30 meter length wrapped as single “layer” or coil, respectively, around a borosilicate cooling tube, here a Duran 50 glass; the inner lamp tube was made out of quartz glass. “Type b” TFAC preparation out of HCFC-123 using Cl₂ as sensitizer and FEP-coil reactor.

A TQ 718 Hg-medium pressure lamp was used with 700 W power in a 400 ml total volume photo reactor and just compressed air with air flow between quartz glass lamp tube and borosilicate cooling tube (just as cooling media for the lamp). Cooling media for the FEP-coil is methanol cooled with a Lauda Cryostat to 30° C. but also other heat transfer media having no (or low) light absorption in the nm range required for photo-oxidation can be used.

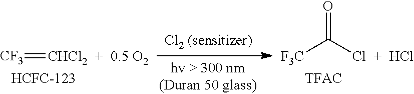

The HCFC-123 (feed 200 g/h) was vaporized (vaporizer oil heated to 100° C.) and mixed with 0.6 mol-% 02 and 10 mol-% Cl₂ before the mixture entered as gas the FEP-coil at the bottom. The observed pressure was atmospheric pressure or slightly above, respectively. Samples were taken out of the gas stream leaving the coil at the top of the reactor. The HCFC-123 conversion was 97%, the selectivity to TFAC taken out of a sample from the gas stream with a so called gas mouse (20 ml sampling device) taken after the EP-coil 99.8%, all values given in GC area % on a CP-SIL 8 column.

### Example 11 (Disclosure)

Photo-oxidation of HCFC-123 to TFAC in photo microreactor.

See FIGS. 6, 7, and 8 for apparatus and reaction.

Apparatus: Dow Corning photo reactor type G1 with an internal volume of 9 ml. (www.corning.com/media/worldwide/Innovation/documents/G1photo_WEB.pdf). The construction of the irradiated glass channels (365 nm LEDs) as shown, e.g., in FIG. 8. The LED power was set to 100 mW/cm².

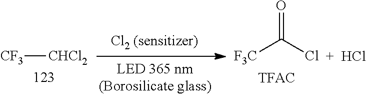

The HCFC-123 (feed 200 g/h), same as above, was vaporized in a vaporizer (vaporizer oil heated to 100° C.) and mixed with 0.6 mol-% O₂ and 10 mol-% Cl₂ before the mixture entered as gas the photo reactor G1. The observed pressure was atmospheric pressure, temperature kept at 35° C. Samples were taken out of the gas stream leaving the reactor. The HCFC-123 conversion was 100%, the selectivity to TFAC taken out of a sample from the gas stream with a gas mouse (20 ml sampling device) taken after the EP-coil 99.8%; all values given in GC area % on a CP-SIL 8 column.

### Example 12 (Disclosure)

Photo-oxidation of HCFC-122 to CDFAC in photo microreactor.

See FIGS. 6, 7 and 8 for apparatus and reaction.

Dow Corning photo reactor type G1 with an internal volume of 9 ml as in Example 11 with 365 nm LEDs. The LED power was set to 100 mW/cm². For example, see FIG. 8.

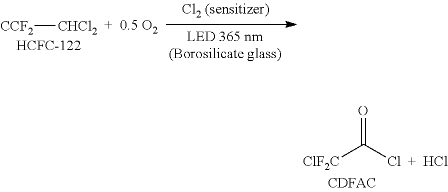

The HCFC-122 (feed 150 g/h) was vaporized in a vaporizer (vaporizer oil heated to 110° C.) and mixed with 0.6 mol-% O₂ and 10 mol-% Cl₂ before the mixture entered as gas the photo reactor G1. The observed pressure was atmospheric pressure, temperature kept at 68° C. Samples were taken out of the gas stream leaving the reactor. The HCFC-122 conversion was 100%, the selectivity to CDFAC taken out of a sample from the gas stream with a gas mouse (20 ml sampling device) taken after the FEP-coil 99.1%; all values given in GC area % on a CP-SIL 8 column.

### Example 13 (Disclosure, Elimination of Oxidizing By-Products)

Conversion of oxidizing material over CuO (copper oxide) to TFAC.

A quantity of 100 g oxidizing material collected out of chlorine sensitized photo-oxidation reaction of HCFC-123 to TFAC was dropped over a period of 1 h to 10 g CuO which was placed in a three-necked flask at room temperature. A gas evolution could be observed which was leaving the flask continuously and which was analyzed by GC-MS to be trifluoroacetyl chloride (TFAC). All the TFAC material was condensed in a dry ice cooling trap. The isolated yield of TFAC was 85%.

### Example 14 (Disclosure, Elimination of Oxidizing By-Products)

Conversion of oxidizing material over NiCl₂ to TFAC.

A quantity of 100 g oxidizing material collected out of chlorine sensitized photo-oxidation reaction of HCFC-123 to TFAC was dropped over a period of 1 h to 10 g NiCl₂ which was placed in a three-necked flask at room temperature without any stirring. A gas evolution could be observed which was leaving the flask continuously and which was analyzed by GC-MS to be 100% trifluoroacetyl chloride (TFAC). All the TFAC material was condensed in a dry ice cooling trap. The isolated yield of TFAC was 65%.

### Example 15 (Disclosure, Elimination of Oxidizing By-Products)

Conversion of oxidizing material over active carbon to TFAC:

A quantity of 50 g oxidizing material collected out of chlorine sensitized photo-oxidation reaction of HCFC-123 to TFAC was dropped over a period of 1 h to 5 g active carbon (supplier Norit, www.norit.com, type RB3) which was placed in a three-necked flask at room temperature without any stirring. A gas evolution with strong exothermic activity could be observed which was leaving the flask continuously and which was analyzed by GC-MS to be 100% trifluoroacetyl chloride (TFAC). All the TFAC material was condensed in a dry ice cooling trap. The isolated yield of TFAC was 99%.

### Example 16 (Disclosure, Elimination of Oxidizing By-Products)

Conversion of oxidizing material over corroded 1.4571 stainless steel

Stainless steel formerly corroded by oxidizing material was cut into 5×5 mm pieces (d=3 mm) and put into a three-necked glass flask. A quantity of 200 g oxidizing material was dropped at room temperature slowly onto this pieces which leads to observed exothermic activity and gas evolution which was identified by GC as TFAC.

### Example 17 (Disclosure, Elimination of Oxidizing By-Products)

Conversion of oxidizing material over pre-treated and deactivated active carbon to TFAC:

A quantity of 50 g active carbon (supplier Norit, www.norit.com, type RB3) was placed into a three-necked flask and slowly flushed with HCFC-123 out of a dropping funnel to reduce activity. An exothermic activity could be observed. Liquid HCFC-123 was decanted from the active carbon, afterwards a quantity of 500 g oxidizing material collected out of chlorine sensitized photo-oxidation reaction of HCFC-123 to TFAC was dropped over a period of 1 h onto the quantity of before pre-treated 50 g active carbon. A gas evolution could be observed which was leaving the flask continuously and which was analyzed by GC-MS to be 100% trifluoroacetyl chloride (TFAC). All the TFAC material was condensed in a dry ice cooling trap. The isolated yield of TFAC was quantitative.

### Example 18 (Disclosure, Elimination of Oxidizing By-Products)

Pre-treated active carbon for usage in industrial TFAC production.

A 301 stainless steel vessel filled with pre-treaded active carbon (supplier Norit, www.norit.com, type RB3; pre-treatment with HCFC-123 as described, e.g., in Example 17) was installed after a 200 l photo reactor (reaction media volume, 3 m length without the electrical head for the lamp) with its exterior made out of Ni, and comprising a 40 kW Hg-medium pressure lamp from Heraeus Noblelight, a cooling tube made out of borosilicate glass, and interior installations (i.e., such as described by the description and the Examples present disclosure).

“Type b” TFAC preparation out of HCFC-123 using Cl₂ as sensitizer was performed in said photo reactor with cooling tube made out of borosilicate glass, the lamp tube was made out of quartz glass, cooling water 25° C., interior Ni-plate every 5 cm.

A quantity of 30 mol HCFC-123/h was vaporized at 60° C. and mixed with 0.52 equivalents of O₂ and 10 mol-% Cl₂ (0.1 equivalents), and this mixture was fed into the photo reactor with exterior made out of nickel metal, and the material leaving the photo reactor was passed over the stainless steel vessel filled with pre-treaded active carbon.

The conversion of HCFC-123 was 89%, the selectivity to TFAC 99.5%. A quantity of 0.2% CFC-113a (1,1,1-trichloro-2,2,2-trifluoroethane) but no phosgenes were detected; all values given in GC area % on a CP-SIL 8 column. No oxidizing material could be observed after the reactor (no condensate at 30° C.). The absence of oxidizing material was confirmed by DSC analysis.

### Example 18 (Comparative Example)

TFAC production without active carbon.

If Example 17 was done without passing over a stainless steel vessel with pre-treaded active carbon, a quantity of 3.5% of oxidizing material (collected in a trap at 20° C.) was found to be present in the gas stream leaving the photo reactor.

### Example 19 (Comparative Example)

CDFAC photo-oxidation without active carbon.

If the equipment as described in Example 17 was used in a HCFC-122 to CDFAC photo-oxidation without passing over a stainless steel vessel with pre-treaded active carbon, a quantity of 1.5% oxidizing material was found (isolated as liquid phase by 60° C.).

### Example 20 (Disclosure)

Pre-treated active carbon for usage in industrial CDFAC production

If Example 19 was performed with pre-treated active carbon none oxidizing material was found.

### Example 21 (Disclosure)

Pre-treated active carbon for usage in industrial production, using a decomposition reactor.

Set up and reaction as described in Example 3. The gas stream after the reactor without feeding over a container (or pipe) with initiating material contained oxidizing material with (estimated) 3.5 wt.-% (DSC analysis). In addition to the described installation out of Example 3, a 20 l volume container filled with (pre-treated with HCFC-123, now deactivated) Norrit RB3 active carbon was installed and the product gas mixture after the reactor was passed through. The oxidizing material content was reduced now to 0% (DSC analysis). For production purpose after decomposition of the oxidizing material, the product gas stream is fed over a membrane compressor^{*)} (Corblin company) into a stainless steel pressure distillation to remove HCl and (traces) other gases as well as recovery of the not consumed Cl₂ applied as sensitizer. The isolated TFAC yield was 99%. *) a piston compressor leads to tars formation and is less suitable

Reference is made to FIGS. 9A and 9B which show a representative scheme of a photochemical reaction with a continuous decomposition procedure exemplified for the manufacture of TFAC. FIG. 9A is showing a reactor sequence with a decomposition reactor; e.g., filled with carbon. FIG. 9B is showing a reactor sequence with a pipe as decomposition reactor; e.g., filled with carbon.

The foregoing description of the exemplary embodiments of the disclosure illustrates and describes the present invention. Additionally, the disclosure shows and describes only the exemplary embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

1. A photochemical reactor comprising:
at least one UV lighting source providing UV lighting emission in a range of short-wavelength UV light to long-wavelength UV light of from about 100 nm to about 400 nm;
a reactor reservoir being surrounded by a reactor wall;
one or more inlet(s)/outlet(s) installation(s);
one or more interior installations each forming a channel inside the reactor reservoir, wherein the one or more interior installations are in tube/pipe form and consist of borosilicate glass and/or of a quartz glass, and wherein each of the interior installations at both tube/pipe ends have an inlet/outlet installation and extending to the exterior of the reactor reservoir; and
optionally, two or more interior installations in plate form inside the reactor reservoir, wherein the two or more interior installations in plate form are horizontally arranged in relation to the one or more interior installations and in relation to a height of the reactor reservoir, and wherein the two or more interior installations in plate form divide the reactor reservoir into a further channel system, each channel starting/ending at one of the one or more inlet(s)/outlet(s) installation(s), respectively.

2. A photochemical reactor comprising:
at least one UV lighting source providing UV lighting emission in a range of short-wavelength UV light to long-wavelength UV light of from about 100 nm to about 400 nm;
a reactor reservoir being surrounded by a reactor wall;
one or more inlet(s)/outlet(s) installation(s); and
one or more interior installations each forming a channel inside the reactor reservoir, wherein the one or more interior installations are in tube/pipe form and consist of borosilicate glass and/or of a quartz glass, and wherein each of the interior installations at both tube/pipe ends have an inlet/outlet installation and extending to the exterior of the reactor reservoir.

3. The photochemical reactor according to claim 2, wherein the one or more interior installations in tube/pipe form are linear or straight, bent, curved, snake shaped, sloped, zigzag or coiled tube/pipe, or combinations thereof.

4. The photochemical reactor according to claim 2, wherein the one or more interior installations are arranged around a mercury-vapor lamp.

5. The photochemical reactor according to claim 2, wherein the one or more interior installations are arranged in a vertical or in a horizontal direction in relation to a bottom surface of the reactor reservoir, and in an upright direction of a mercury-vapor lamp inside the reactor reservoir.

6. The photochemical reactor according to claim 5, wherein the one or more interior installations are arranged inside the reactor reservoir in the vertical direction in relation to the bottom surface of the reactor reservoir.

7. The photochemical reactor according to claim 6, wherein the one or more interior installations are in a linear or straight tube/pipe form.

8. The photochemical reactor according to claim 7, wherein the one or more interior installations are connected at their outside with each other and/or held in place by one or more horizontal plates.

9. The photochemical reactor according to claim 4, wherein the interior installation is a single interior installation in the coiled tube/pipe form, wherein the single interior installation is installed in the upright direction, and wherein the single interior installation is surrounding the mercury-vapor lamp.

10. The photochemical reactor according to claim 2, wherein the reactor reservoir is a photochemical reaction zone and the interior installation is a cooling medium zone.

11. The photochemical reactor according to claim 10, wherein the one or more interior installations are connected at their outside with each other and/or held in place by one or more horizontal plates.

12. The photochemical reactor according to claim 2, wherein the interior installation is a photochemical reaction zone and the reactor reservoir is a a cooling medium zone.

13. The photochemical reactor according to claim 12, wherein the interior installation is a single interior installation in the coiled tube/pipe form, wherein the single interior installation is installed in the upright direction, and wherein the single interior installation is surrounding the mercury-vapor lamp.

14. A process for performing a photochemical oxidation of an organic compound, wherein the organic compound is reacted with oxygen in a gas phase by light induced photochemical oxidation by:
a) a direct gas phase photochemical oxidation with short UV wavelength (λ) of greater than 200 nm, but up to no more than 400 nm, or by
b) a sensitized gas phase photochemical oxidation with addition of elemental chlorine as sensitizer/light absorber for activating irradiation with light of UV wavelength (λ) of greater than 290 nm, but up to no more than 400 nm, of a mercury-vapor lamp;
and wherein the light induced photochemical oxidation is performed in a photochemical reactor according to claim 1.

15. A process for manufacturing a polyfluorochloro- and/or perfluorocarboxylic acid chloride compound of formula R^{a}CR^{b}XC(O)Cl, wherein R^{a} is chlorine, fluorine or perfluorinated alkyl having 1 to 10 carbon atoms, and R^{b} is hydrogen, chlorine or, fluorine, and X is chlorine or fluorine, by performing a photochemical oxidation of an organic compound selected from the group consisting of
(i) a halogeno-alkane compound of formula R¹CFXCHCl₂, wherein R¹ is fluorine, chlorine or perfluorinated alkyl having 1 to 10 carbon atoms, and X is chlorine or fluorine, and
(ii) a halogeno-alkylene compound of formula R²CX═CHₙClₘ, wherein R² is fluorine, chlorine or perfluorinated alkyl having 1 to 10 carbon atoms, and X is chlorine or fluorine, and n is an integer of 0 to 1, and m is an integer of 1 to 2, and a sum of n and m is 2;
wherein
the organic compound as defined in (i) or (ii) is reacted with oxygen in the gas phase by light induced photochemical oxidation by
a) direct gas phase photochemical oxidation with short UV wavelength (λ) of greater than 200 nm, but up to no more than 400 nm, or by
b) sensitized gas phase photochemical oxidation with addition of elemental chlorine as sensitizer/light absorber for activating irradiation with light of the UV wavelength (λ) of greater than 290 nm of a mercury-vapor lamp;
and wherein the light induced photochemical oxidation is performed in a photochemical reactor according to claim 1.

16. The process for manufacturing the polyfluorochloro- and/or perfluorocarboxylic acid chloride compound according to claim 14, wherein in the compound of formula R^{a}CR^{b}XC(O)Cl manufactured by performing the photochemical oxidation the substituent R^{a} is chlorine or fluorine, and R^{b} is hydrogen, chlorine or, fluorine, and X is chlorine or fluorine.

17. The process for manufacturing the polyfluorochloro- and/or perfluorocarboxylic acid chloride compound according to claim 16, wherein the compound of formula R^{a}CR^{b}XC(O)Cl manufactured by performing the photochemical oxidation is selected from the group consisting of trifluoroacetyl chloride, chlorodifluoroacetyl chloride, trichloroacetyl chloride, and dichloroacetyl chloride.

18. The process for manufacturing the polyfluorochloro- and/or perfluorocarboxylic acid chloride compound of formula R^{a}CR^{b}XC(O)Cl, according to claim 15, wherein the organic compound subjected to performing the photochemical oxidation is selected from the group consisting of
(i) the halogenoalkane compound of formula R¹CFXCHCl₂, wherein the substituent R¹ is fluorine or chlorine, and X is chlorine or fluorine, and
(ii) the halogenoalkylene compound of formula R²CX═CHₙClₘ, wherein the substituent R² is fluorine or chlorine, and X is chlorine or fluorine, and n is an integer of 0 to 1, and m is an integer of 1 to 2, and the sum of n and m is 2.

19. The process for manufacturing a polyfluorochloro- and/or perfluorocarboxylic acid chloride compound of formula R^{a}CR^{b}XC(O)Cl, according to claim 18, wherein
(i) the halogenoalkane compound of formula R¹CFXCHCl₂ subjected to performing the photochemical oxidation is selected from the group consisting of 1,1,1-trifluoro-2,2-dichloroethane and 1,1-difluoro-1,2,2-trichloroethane; or
(ii) the halogeno-alkylene compound of formula R²CX═CHₙClₘ subjected to performing the photochemical oxidation is selected from the group consisting of 1,1-dichloro-2-chloroethylene and perchloroethylene.

20. The process for manufacturing a polyfluorochloro- and/or perfluorocarboxylic acid chloride compound of formula R^{a}CR^{b}XC(O)Cl, according to claim 19, wherein
(i) the halogenoalkane compound of formula R¹CFXCHCl₂ subjected to performing the photochemical oxidation is
(i-1) 1,1,1-trifluoro-2,2-dichloroethane and the compound manufactured by the photochemical oxidation thereof having a R^{a}CR^{b}XC(O)Cl is trifluoroacetyl chloride; or
(i-2) 1,1-difluoro-1,2,2-trichloroethane and the compound manufactured by the photochemical oxidation thereof having formula R^{a}CR^{b}XC(O)Cl is chlorodifluoroacetyl chloride;
or
(ii) the halogeno-alkylene compound of formula R²CX═CHₙClₘ subjected to performing the photochemical oxidation is
(ii-1) 1,1-dichloro-2-chloroethylene and the compound manufactured by the photochemical oxidation thereof having formula R^{a}CR^{b}XC(O)Cl is dichloroacetyl chloride; or
(ii-1) perchloroethylene and the compound manufactured by the photochemical oxidation thereof having formula R^{a}CR^{b}XC(O)Cl is trichloroacetyl chloride.

21. A process for performing a photochemical oxidation of an organic compound, wherein the organic compound is reacted with oxygen in a gas phase by light induced photochemical oxidation, wherein the light induced photochemical oxidation is performed in a photochemical microreactor comprising:
at least one array of UV lighting sources providing UV lighting emission in a range of short-wavelength UV light to long-wavelength UV light of from about 100 nm to about 400 nm, and wherein a UV wavelength (λ) is configured for direct gas phase photochemical oxidation or for sensitized gas phase photochemical oxidation, or is or adjustable thereto;
at least one reactor reservoir surrounded by a reactor wall;
one or more inlet(s)/outlet(s) installation(s) and;
and one or more interior installations each forming a channel inside the reactor reservoir, wherein the one or more interior installations are in tube/pipe form and consist of glass, and wherein each of the interior installations at both tube/pipe ends have an inlet/outlet installation and extending to the exterior of the reactor reservoir.

22. A process for performing a photochemical oxidation of an organic compound, wherein the organic compound is reacted with oxygen in the gas phase by light induced photochemical oxidation by:
a) a direct gas phase light induced photochemical oxidation with UV wavelength (λ) of greater than 200 nm and mainly about 365 nm line provided by at least one LED UV lighting source, or by:
b) a sensitized gas phase photochemical oxidation with addition of elemental chlorine as sensitizer/light absorber for activating irradiation with light of UV wavelength (λ) of greater than 290 nm, provided by at least one UV lighting source with UV wavelength (λ) of mainly about 365 nm line of LED UV lighting source;
and wherein the light induced photochemical oxidation is performed in a photochemical microreactor comprising:
at least one array of UV lighting sources providing UV lighting emission in a range of short-wavelength UV light to long-wavelength UV light of from about 100 nm to about 400 nm, or of from about >290 nm to about 400 nm, or of from about >300 nm to about 400 nm, and wherein the UV wavelength (λ) is adjusted to the UV wavelength (λ) for the direct gas phase photochemical oxidation or to the UV wavelength (λ) for the sensitized gas phase photochemical oxidation, or is adjustable thereto;
at least one reactor reservoir surrounded by a reactor wall;
one or more inlet(s)/outlet(s) installation(s); and
one or more interior installations each forming a channel inside the reactor reservoir, wherein the one or more interior installations are in tube/pipe form and consist of glass, and wherein each of the interior installations at both tube/pipe ends have an inlet/outlet installation and extending to the exterior of the reactor reservoir.

23. The process for performing a photochemical oxidation of an organic compound, wherein the organic compound is reacted with oxygen in the gas phase by light induced photochemical oxidation, according to claim 21, wherein the one or more interior installations each form a channel and the at least one array of UV lighting sources together form an irradiated glass channel system.

24. The process for performing a photochemical oxidation of an organic compound, wherein the organic compound is reacted with oxygen in the gas phase by light induced photochemical oxidation, according to claim 16 for oxidizing material(s) and/or oxidizing by-product(s) formed in the photochemical oxidation,
wherein the oxidizing material(s) and/or oxidizing by-product(s) are an organic peroxide compound derived from the organic compound that is reacted with oxygen in the gas phase by the light induced photochemical oxidation,
is eliminated from the reaction mixture obtained from the photochemical oxidation by contacting and/or treating the oxidizing material and/or the oxidizing by-products with initiating material(s).

25. The process for performing a photochemical oxidation of an organic compound, wherein the organic compound is reacted with oxygen in the gas phase by the light induced photochemical oxidation according to claim 24, wherein the initiating material(s):
(i) is/are a radical or free radical generating agent, and/or
(ii) is/are selected from the group consisting of carbon black steel or carbon steel; corroded metals; transition metal oxides; wherein the metal is selected from the group consisting of iron, cobalt, copper, nickel, tungsten, and zinc; transition metal chlorides, wherein the transition metal is selected from the group consisting of iron, cobalt, copper, nickel, tungsten, and zinc, mixtures of the transition metal oxides, transition metal chlorides, and/or mixtures of the transition metal oxides and chlorides; carbon, activated carbon, deactivated active carbon, and pre-treated active carbon.

26. The process for performing a photochemical oxidation of an organic compound, wherein the organic compound is reacted with oxygen in the gas phase by the light induced photochemical oxidation, according to claim 25, wherein the initiating material(s) is selected from the group consisting of carbon black steel or carbon steel; corroded steel; iron oxide(s), cobalt oxide(s), copper oxide(s), nickel oxide(s), tungsten oxide(s), zinc oxide(s), iron chloride(s), cobalt chloride(s), copper chloride(s), nickel chloride(s), tungsten chloride(s), and zinc chloride(s); mixtures of the oxides, chlorides and/or mixtures of the oxides and chlorides; carbon, activated carbon, deactivated active carbon, and pre-treated active carbon.

27. The process for performing a photochemical oxidation of an organic compound, wherein the organic compound is reacted with oxygen in the gas phase by the light induced photochemical oxidation according to claim 25,
wherein the oxidizing material(s) and/or oxidizing by-product(s) is/are an organic peroxide compound derived from the organic compound that is reacted with oxygen in the gas phase by the light induced photochemical oxidation,
is eliminated from the reaction mixture obtained from the photochemical oxidation of the process according to claim 15 for manufacturing a polyfluorochloro- and/or perfluorocarboxylic acid chloride compound of formula R^{a}CR^{b}XC(O)Cl according to claim 15.

28. The process for performing a photochemical oxidation of an organic compound, wherein the organic compound is reacted with oxygen in the gas phase by the light induced photochemical oxidation according to claim 27, wherein the organic compound selected from the group consisting of
(i) a halogeno-alkane compound of formula R¹CFXCHCl₂, wherein R¹ is fluorine, chlorine or perfluorinated alkyl having 1 to 10 carbon atoms, and X is chlorine or fluorine, and
(ii) a halogeno-alkylene compound of formula R²CX═CHₙClₘ, wherein R² is fluorine, chlorine or perfluorinated alkyl having 1 to 10 carbon atoms, and X is chlorine or fluorine, and n is an integer of 0 to 1, and m is an integer of 1 to 2, and a sum of n and m is 2.

29. The process for performing a photochemical oxidation of an organic compound, wherein the organic compound is reacted with oxygen in the gas phase by the light induced photochemical oxidation, according to claim 27, wherein in the compound of formula R^{a}CR^{b}XC(O)Cl manufactured by performing the photochemical oxidation the substituent R^{a} is chlorine or fluorine, and R^{b} is hydrogen, chlorine or, fluorine, and X is chlorine or fluorine; or
wherein the compound of formula R^{a}CR^{b}XC(O)Cl manufactured by performing the photochemical oxidation is selected from the group consisting of trifluoroacetyl, chlorodifluoroacetyl chloride, trichloroacetyl chloride, and dichloroacetyl chloride.

30. The process for performing a photochemical oxidation of an organic compound, wherein the organic compound is reacted with oxygen in the gas phase by light induced photochemical oxidation, according to claim 28, wherein the organic compound is (i) a halogeno-alkane compound of formula R¹CFXCHCl₂ according to claim 18 and/or (ii) a halogeno-alkylene compound of formula R²CX═CHₙClₘ according to claim 18.

31. The process for performing a photochemical oxidation of an organic compound, wherein the organic compound is reacted with oxygen in the gas phase by the light induced photochemical oxidation according to claim 14, wherein the process further comprises passing the reaction mixture obtained by the photochemical reaction over a decomposition reactor, optionally a decomposition reactor filled with carbon.
